(19) United States
(12) Patent Application Publication
(10) Pub. No.: US 2023/0100306 A1
(43) Pub. Date: Mar. 30, 2023

(54) SCALABLE BIOREACTOR SYSTEMS AND RELATED METHODS OF USE

(71) Applicant: Mission Barns, Inc., Berkeley, CA (US)

(72) Inventors: George C. ENGELMAYR, Jr., Emeryville, CA (US); Ilan Eitan FISCHER, Berkeley, CA (US); Andre BERGERON, San Francisco, CA (US); Saam SHAHROKHI, Oakland, CA (US); William C. PATON, San Francisco, CA (US); Viktor MACIAG, Berkeley, CA (US); Xiao Guan RADSTROM, Berkeley, CA (US); Amelia BEVILLE, Berkeley, CA (US); Robert YAMAN, Berkeley, CA (US)

(21) Appl. No.: 17/901,573

(22) Filed: Sep. 1, 2022

Related U.S. Application Data
(63) Continuation of application No. PCT/US2021/026071, filed on Apr. 6, 2021.
(60) Provisional application No. 63/005,979, filed on Apr. 6, 2020.

(51) Int. Cl. C12M 3/06; C12M 1/12; C12M 1/00; C12M 1/34

(52) U.S. Cl. CPC C12M 27/14; C12M 25/04; C12M 23/20; C12M 23/06; C12M 41/42

(57) ABSTRACT

Bioreactors configured to scale-up the production of greater quantities of cells at relatively low cost are provided. These bioreactors may be utilized in the production of large-scale quantities of cell-based meat and cell-based fat. The bioreactors may be reusable and may have a high surface area-to-volume ratio for adherent cell expansion. The bioreactors may be capable of yielding a large number of adherent cells per bioreactor unit.

### CROSS-REFERENCE

This application is a continuation of International Application No. PCT/US2021/026071, filed Apr. 6, 2021, which claims the benefit of U.S. Provisional Application No. 63/005,979, filed Apr. 6, 2020, both of which are incorporated herein by reference in their entirety for all purposes.

### BACKGROUND

Bioreactors for animal cell proliferation, differentiation, and tissue formation (e.g., tissue engineering) typically comprise a sterilizable fluid container, a gas exchanger, and controllers for temperature, pH, and humidity/evaporation. For example, a sterile polystyrene Petri dish with a vented lid in a CO₂ incubator can be considered a simple bioreactor system.

Animal cell cultures within bioreactors may take the form of suspension cultures, adherent cultures, or combinations of the two (e.g., microcarrier culture, etc.). Suspension cultures are generally characterized by relatively non-adherent animal cell cultures and generally characterized by single cells or cell aggregates (e.g., colonies) having the capacity to adhere to, proliferate on, and differentiate on a cell-culture substrate (e.g., tissue culture-treated polystyrene).

Cell-culture substrates utilized in adherent animal cell culture may take many forms, including those employed in so-called “2-D culture,” wherein cell-culture substrates generally comprise substantially smooth-to-the-naked-eye materials having surface properties (e.g., hydrophilicity) amenable to cell-adhesive protein adsorption (e.g., fibronectin from fetal bovine serum) and thereby cell adhesion (e.g., via integrin-mediated binding to cell-adhesive peptide sequences presented by the adsorbed proteins (e.g., the arginine-glycine-aspartic acid (RGD) sequence in fibronectin)). Cell-culture substrates may be substantially non-degradable, as in the case of polystyrene, or substantially degradable, as in the case of a variety of bioresorbable polymers utilized as tissue-engineering scaffolds (e.g., poly-glycolic acid (PGA), poly-L-lactic acid (PLLA), etc.).

Bioreactors utilized in industrial-scale cell culture or tissue engineering typically provide additional features beyond those of lab or bench-scale bioreactors, including fluid mixers (e.g., an agitator to entrain gas into the culture media), a temperature controller (e.g., a heating jacket), and sterilizable ports for periodic and/or continuous liquid and gas exchange. Industrial-scale bioreactors also typically employ instrumentation for process monitoring and control, such as, for example, temperature sensors, dissolved oxygen and carbon dioxide sensors, pH sensors, glucose and/or lactate sensors, and optical density sensors. Bioreactor process control is often implemented via a combination of gas mass-flow controllers, dosing pumps (e.g., for acid, base, and/or anti-foam), automated valves, and agitator speed controllers. Computer control loop algorithms (e.g., proportional integral derivative (PID) control) can employ sensor feedback, comparing process variable measurements to set points, and send control signals to adjust process parameters (e.g., for the control of dissolved gas concentrations and pH).

Bioreactors utilized in tissue engineering often include additional features beyond the aforementioned features typical of more conventional animal cell culture bioreactors. For example, tissue-engineering bioreactors often include components or elements for imparting biophysical stimuli to the forming tissues and/or organs (e.g., linear actuators and grips to provide cyclic stretch and/or flexure, pumps to provide fluid shear stress, and electrodes to provide electrical stimulation). Such biophysical stimuli have been demonstrated, in many cases, to be capable of mediating a broad spectrum of tissue formation and remodeling phenomena, including, for example, the mechanical activation of cellular differentiation (i.e., mechanotransduction) and compaction and alignment of the extracellular matrix.

### SUMMARY

Recognized herein is the need for bioreactors capable of scale-up to the production of greater quantities of cells at relatively low cost and using fewer resources (e.g., source or starting materials). These bioreactors may be utilized in the production of large-scale quantities of cell-based meat and/or cell-based fat. The bioreactors can be substantially reusable (other than routine cleaning, sterilization, and maintenance) and have a relatively high surface area-to-volume ratio for adherent cell expansion to minimize the cost of producing the cells. The bioreactors can be capable of yielding a relatively large number of adherent animal cells per bioreactor unit. For example, a scalable bioreactor system for cell-based meat and/or cell-based fat production can include a relatively high surface area of cell-culture substrate per unit volume of the bioreactor system.

The present disclosure provides bioreactors and methods of operation thereof that may enable the efficient growth of cells. The bioreactors of the present disclosure and the associated methods of use or operation thereof may yield a greater amount of cell production while minimizing a surface area required to cultivate the cells. The bioreactors may generally comprise a container containing a rotating assembly of a plurality of porous or semi-permeable cell-culture substrates. The substrates are generally rotated through a fluid interface such as an air interface, a liquid interface, or an air-liquid (e.g., air-culture medium) interface to provide enhanced fluid transport (e.g., liquid and/or gas transport) to and from the adherent cells during each pass through the gas phase, while incurring minimal fluid shear stresses during each pass through and nutrient exchange with the liquid phase.

In some cases, a bioreactor may comprise a plurality of alternating growth substrates and spacer substrates. A ratio of an area of the growth substrates to an area of the spacer substrates may be 1000:1 to 1:1 (e.g., 900:1 to 1:1, 800:1 to 1:1, 400:1 to 1:1, 300:1 to 1:1, 200:1 to 1:1, or another suitable ratio). In certain cases, the ratio of the area of the growth substrates to the area of the spacer substrates may be 200:1 to 2:1 (e.g., 200:1 to 2:1, 175:1 to 2:1, 150:1 to 2:1, 125:1 to 2:1, 100:1 to 2:1, 75:1 to 2:1, 50:1 to 2:1, 25:1 to 2:1, or another suitable ratio).

The bioreactor may include a container. The container may be configured to be partially filled with a growth medium such that at a given time point a first portion of each of the substrates is disposed in the growth medium and a second portion of the substrates is not disposed in the growth medium. The growth substrates may comprise a mesh. The mesh of the growth substrate may be at least a 10×10 mesh size (e.g., at least a 12×12, 16×16, 25×25, 50×50, 75×75, 100×100, or another suitable mesh size). The spacer substrates may comprise a mesh. The mesh of the spacer substrates may be at least a 1×1 mesh size (e.g., at least a 2×2, 3×3, 5×5, 10×10, 20×20, 50×50, or another suitable mesh size). In some cases, the growth substrates may have an average pore size greater than an average pore size of the spacer substrates.

In certain cases, the plurality of cell-culture substrates may be formed from a material comprising stainless steel (e.g., 304L stainless steel, 316 stainless steel, 430 stainless steel, etc.), titanium, etc. The plurality of cell-culture substrates may be formed from a material comprising borosilicate glass, titanium dioxide ceramic, etc. The plurality of cell-culture substrates may be formed from a material comprising a corrosion-resistant metal, a corrosion-resistant metal alloy, etc. The plurality of cell-culture substrates may be formed from a material comprising a polymer, Ultem™ 1010, polyethylene terephthalate, poly(glycolic acid) (PGA), poly(L-lactic acid) (PLLA), poly(lactide-co-glycolide) (PLGA), polycaprolactone (PCL), polyglycerol sebacate (PGS), etc. The plurality of cell-culture substrates may be formed from a material comprising a sugar-based material, a carbohydrate-based material, a protein-based material, a fat-based material, etc. The plurality of cell-culture substrates may be formed from a material comprising pectin, chitosan, a grain-based product (e.g., a rice-based product, a corn-based product, a wheat-based product, etc.), a seaweed-based product, an algae-based product, etc. The plurality of cell-culture substrates may be formed from a material comprising a degradable material, an edible material, etc. The plurality of cell-culture substrates may be formed from a material comprising a combination of any of the materials provided herein.

The growth substrates may be formed from a first material and the spacer substrates may be formed from a second material. The first material may be different than the second material (e.g., the first material may be a metal and the second material may be a polymer). In some instances, the growth substrates may comprise a coating that adheres or selectively adheres to the cells. The coating may comprise a cell-adhesive protein, a cell-adhesive peptide, a coating derived from an animal cell extracellular matrix, collagen, fibronectin, laminin, poly-L-lysine, poly-D-lysine, a coating derived from a plant, arginine-glycine-aspartic acid (RGD)-containing vitronectin-like protein, or a combination thereof.

Each of the growth substrates may be located at a distance of 0 μm to 5,000 μm from an adjacent spacer substrate (e.g., 1 μm to 5,000 μm, 10 μm to 5,000 μm, 100 μm to 5,000 μm, 1,000 μm to 5,000 μm, 2,000 μm to 5,000 μm, 3,000 μm to 5,000 μm, 4,000 μm to 5,000 μm, 1 μm to 4,000 μm, 1 μm to 3,000 μm, 1 μm to 2,000 μm, 1 μm to 1,000 μm, 1 μm to 750 μm, 1 μm to 500 μm, 1 μm to 250 μm, 1 μm to 100 μm, 1 μm to 10 μm, or any suitable distance).

In certain cases, the growth substrates may be arranged parallel, or substantially parallel, to the spacer substrates. A first growth substrate may be arranged at an angle of 5 degrees to 45 degrees to an adjacent spacer substrate of the spacer substrates (e.g., 5 degrees to 40 degrees, 5 degrees to 30 degrees, 5 degrees to 25 degrees, 5 degrees to 20 degrees, 5 degrees to 15 degrees, 5 degrees to 10 degrees, 10 degrees to 45 degrees, 15 degrees to 45 degrees, 20 degrees to 45 degrees, 25 degrees to 45 degrees, 30 degrees to 45 degrees, 35 degrees to 45 degrees, 40 degrees to 45 degrees, or any suitable angle).

In various cases, the bioreactor may comprise a shaft. The shaft may be coupled to the plurality of cell-culture substrates. The bioreactor may comprise a motor. The motor may be coupled to the shaft, for example, to rotate the shaft, thereby rotating the plurality of cell-culture substrates. The motor may comprise a servomotor or a stepper motor. In some cases, the shaft may be coupled to a lateral center of the growth substrates. The shaft may be coupled to a lateral center of the spacer substrates.

In some embodiments, the growth substrates may be for the growth of a biological material. The biological material may comprise cells, tissues, organs, cell-based meat, cell-based fat, or a combination thereof. A lateral area of a growth substrate may comprise a shape selected from the group consisting of a circle, ellipse, rectangle, square, diamond, triangle, and polygon. A lateral area of a spacer substrate may comprise a shape selected from the group consisting of a circle, ellipse, rectangle, square, diamond, triangle, and polygon. At least a portion of an interior of the container may conform to a shape of the growth substrates and/or a shape of the spacer substrates.

In certain embodiments, the container may comprise a sealable container. The sealable container may comprise a hermetically sealable container. The container may isolate the growth medium from an environment surrounding the container.

In various embodiments, a cell cultivation system may include at least one growth substrate for growing one or more cell types. The at least one growth substrate may have a mesh porosity and a surface area such that a higher cell growth density per unit area is achieved when using a growth substrate that has a higher mesh porosity and a smaller surface area unit area than another growth substrate.

The mesh porosity and the surface area per unit area may be based on a number of openings in the at least one growth substrate. The openings may be arranged in a repeating pattern across a surface of the at least one growth substrate. In some instances, the cell growth density per unit area on the at least one growth substrate may increase with the number of openings. The cell growth density per unit area on the at least one growth substrate may increase by at least 5% when the number of openings is increased by at least 10%.

In various cases, the cell growth density per unit area on the at least one growth substrate may increase with decreasing surface area per unit area. The cell growth density per unit area on the at least one growth substrate may increase by at least 5% when the surface area per unit area is decreased by at least 10%.

In certain cases, the system may include at least one spacer substrate adjacent to the at least one growth substrate. The at least one spacer substrate may be used to facilitate transport and distribution of a growth medium over or across a surface of the at least one growth substrate.

In some embodiments, a cell cultivation system may include at least one growth substrate for growing one or more cell types and at least one spacer substrate adjacent to the at least one growth substrate. The at least one spacer substrate may be sized to influence a cell growth density on the at least one growth substrate, such that a higher cell growth density per unit area is achieved on the at least one growth substrate when using a smaller spacer substrate compared to a larger spacer substrate.

In certain embodiments, a size of the at least one spacer substrate may be based on a dimension as measured along a radial or longitudinal direction of the at least one spacer substrate. The cell growth density per unit area on the at least one growth substrate may increase as the dimension of the at least one spacer substrate is reduced along the radial or longitudinal direction. The cell growth density per unit area on the at least one growth substrate may increase by at least 5% when the size of the at least one spacer substrate is increased by at least 10%.

In various embodiments, the smaller spacer substrate may have a smaller surface area than the larger spacer substrate. The smaller spacer substrate may have a same thickness, or substantially same thickness, as the larger spacer substrate. A size of the at least one growth substrate may be substantially equal to or greater than a size of the at least one spacer substrate. The at least one growth substrate and the at least one spacer substrate may comprise a same number of openings per unit area. The at least one growth substrate and the at least one spacer substrate may each comprise a different number of openings per unit area.

In some instances, a bioreactor may comprise a container, a rotatable shaft disposed in the container, and a first growth substrate comprising a growth surface and a second growth substrate comprising a growth surface. The growth surface may be for growth of a biological material, and the first growth substrate and the second growth substrate may be coupled to the rotatable shaft. The bioreactor may comprise a first spacer substrate disposed between the first growth substrate and the second growth substrate.

The container may be configured to retain a growth medium. In some cases, the growth surface may be substantially planar. Each of the first growth substrate and the second growth substrate may comprise a mesh. The mesh may be formed from a plurality of wires. Each wire of the plurality of wires may have a thickness of 10 μm to 5,000 μm (e.g., 10 μm to 4,000 μm, 10 μm to 3,000 μm, 10 μm to 2,000 μm, 10 μm to 1,000 μm, 10 μm to 750 μm, 10 μm to 500 μm, 10 μm to 250 μm, 10 μm to 100 μm, 50 μm to 5,000 μm, 100 μm to 5,000 μm, 250 μm to 5,000 μm, 500 μm to 5,000 μm, 1,000 μm to 5,000 μm, 2,000 μm to 5,000 μm, 3,000 μm to 5,000 μm, 4,000 μm to 5,000 μm, or any other suitable thickness).

Adjacent wires of the plurality of wires may be separated by a distance of 1 μm to 5,000 μm (e.g., 1 μm to 4,000 μm, 1 μm to 3,000 μm, 1 μm to 2,000 μm, 1 μm to 1,000 μm, 1 μm to 750 μm, 1 μm to 500 μm, 1 μm to 250 μm, 1 μm to 100 μm, 10 μm to 5,000 μm, 25 μm to 5,000 μm, 50 μm to 5,000 μm, 100 μm to 5,000 μm, 500 μm to 5,000 μm, 1,000 μm to 5,000 μm, 2,000 μm to 5,000 μm, 3,000 μm to 5,000 μm, 4,000 μm to 5,000 μm, or any other suitable distance).

In some cases, each of the first growth substrate and the second growth substrate may be porous. The first growth substrate and the second growth substrate may have a porosity of 0.001 to 0.999 (e.g., 0.01 to 0.99, 0.01 to 0.999, 0.1 to 0.999, 0.25 to 0.999, 0.25 to 0.999, 0.50 to 0.999, 0.75 to 0.999, 0.90 to 0.999, or any other suitable porosity). The first growth substrate may be disposed 0 μm to 5,000 μm from the first spacer substrate (e.g., 1 μm to 5,000 μm, 10 μm to 5,000 μm, 100 μm to 5,000 μm, 1,000 μm to 5,000 μm, 2,000 μm to 5,000 μm, 3,000 μm to 5,000 μm, 4,000 μm to 5,000 μm, 1 μm to 4,000 μm, 1 μm to 3,000 μm, 1 μm to 2,000 μm, 1 μm to 1,000 μm, 1 μm to 750 μm, 1 μm to 500 μm, 1 μm to 250 μm, 1 μm to 100 μm, 1 μm to 10 μm, or any suitable distance).

The bioreactor may include a plurality of growth substrates and a plurality of spacer substrates. The plurality of growth substrates may be arranged substantially parallel to the plurality of spacer substrates. In some instances, the biological material may comprise cells, tissues, organs, cell-based meat, cell-based fat, or a combination thereof.

In certain instances, the rotatable shaft may be coupled to the first growth substrate and the second substrate. The rotatable shaft may be coupled to a lateral center of the first growth substrate and to a lateral center of the second growth substrate. The bioreactor may comprise a motor coupled to the rotatable shaft to rotate the rotatable shaft, thereby rotating the first growth substrate and the second growth substrate. The motor may comprise a servomotor or a stepper motor.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material

### BRIEF DESCRIPTION OF THE DRAWINGS

The novel features of the systems and methods of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 shows an embodiment of a bioreactor.

FIG. 2 shows an embodiment of a bioreactor in which disk stack rotation is driven via electromagnets.

FIG. 3 illustrates the concept of using electromagnets to drive disk stack rotation in the bioreactor of FIG. 2.

FIG. 4 shows an embodiment of a bioreactor in which disk stack rotation is driven via a directly coupled motor which is parallel to the disk stack.

FIG. 5 shows a photo of an embodiment of a bioreactor in which disk stack rotation is driven via a directly coupled motor which is parallel to the disk stack, as illustrated in FIG. 4.

FIG. 6 shows an embodiment of a bioreactor in which disk stack rotation is driven via a motor attached to the lid of the bioreactor through the use of beveled gears.

FIG. 7 shows embodiments of configurations of cell-culture substrates.

FIG. 8 shows an example of a first method for mimicking manual tapping utilized in harvesting cells from the surface of a tissue culture flask and thereby facilitating cell harvesting (panel A). FIG. 8 also shows an example of a second method for mimicking manual tapping utilized in harvesting cells from the surface of a tissue culture flask and thereby facilitating cell harvesting (panel B).

FIG. 9 shows an embodiment of a cell-culture substrate (panel A). Panel B shows an example configuration of interleaved or alternating “growth disks” and “spacer disks” on a shaft. Panel C shows periodic gas exchange and nutrient exchange with each rotation of a stack of a plurality of interleaved growth disks and spacer disks through culture media partially filling the inside of a container.

FIG. 10 shows a photograph of an embodiment of a bioreactor disclosed herein.

FIG. 11 shows a photo from a substantially top-view of an embodiment of a bioreactor system disclosed herein.

FIG. 12 shows an example phase-contrast light photomicrograph of a 72-hour culture of duck-derived fibroblast-like cells growing into the openings of a cell-culture substrate described herein (panel A). Panel B shows a higher magnification of one opening, in which individual cells are more readily distinguishable within the opening.

FIG. 13 shows an example fluorescence micrograph of a 96-hour culture of duck-derived fibroblast-like cells growing on the wires and into the openings of a cell-culture substrate described herein.

FIG. 14 shows a photo of four different sizes of embodiments of bioreactors disclosed herein.

FIG. 15 is a graph showing an example of glucose consumption rate versus culture time data collected during operation of an embodiments of a bioreactor disclosed herein.

FIG. 16 shows an example of cell density per unit area of cell-culture substrate as a function of culture time collected during operation of an embodiment of a bioreactor disclosed herein (panel A). Panel B shows an example of porcine adipose-derived cells growing into the openings of a cell-culture substrate as a function of time collected during operation of an embodiment of a bioreactor disclosed herein.

FIG. 17 shows an embodiment of a cell-culture substrate wherein additional larger openings are incorporated to provide additional pathways for nutrient and gas exchange.

FIG. 18 shows an embodiment of a bioreactor container in which the inside of the container substantially conforms to the shape and size of a plurality of cell-culture substrates.

FIG. 19 shows an example of a semi-continuous process for the production of cells using a bioreactor as described herein (panel A). In this example, differential cell seeding and proliferation densities can allow for selective cell differentiation and harvest. Panel B illustrates how cell density varies along the horizontal axis of the bioreactor described in panel A.

FIG. 20 shows another example of a semi-continuous process for the production of cells using a bioreactor as described herein. In this example, differential inhibition of adipocyte or adipocyte-like cell differentiation can allow for selective adipocyte or adipocyte-like cell differentiation and harvest.

FIG. 21 is a graph showing an inverse relationship between cell growth density and mesh porosity, in accordance with some embodiments.

FIG. 22 is a graph showing an inverse relationship between cell growth density and the size of the spacer substrate, in accordance with some embodiments.

FIG. 23 is a graph showing that cell growth yields are not significantly sensitive to changes in rotational speeds ranging from about 1 rotation per minute (RPM) to about 3 RPM.

### DETAILED DESCRIPTION

Described herein are bioreactors for growing cells, tissues, organs, cell-based meat, and/or cell-based fat (e.g., cell or tissue-engineering bioreactor systems). The bioreactors include components and configurations of the components (e.g., alternating or interleaving growth substrates and spacer substrates) such that the cells, tissues, etc. can be grown at higher densities than in some other bioreactors. The bioreactor can include a plurality of cell-culture substrates disposed in a container. The cell-culture substrates can include one or more growth substrates (e.g., growth disks) and one or more spacer substrates (e.g., spacer disks).

The growth substrates can include a surface configured or treated for growth of a biological material (e.g., cells, tissues, organs, cell-based meat, cell-based fat, or a combination thereof). The biological material may include muscle cells, connective tissue cells, fat cells, stem cells, mesenchymal cells, vascular cells, blood-associated cells, liver cells, nervous system cells, or cells from any edible animal organ, etc. The cells can be derived from any suitable animal (e.g., a mammal, bird, reptile, amphibian, fish, invertebrates (e.g., insects, mollusks, etc.), etc.). For example, the cells may be bovine, porcine, duck, chicken, alligator, frog, salmon, shark, grasshopper, clam, etc.

The spacer substrates can alternate or be interleaved with the growth substrates (e.g., along a (rotatable) shaft).

The container can be at least partially filled with a growth medium (e.g., a solid, liquid, gas, or vapor growth medium). The growth medium may only partially cover the plurality of cell-culture substrates (e.g., 10%, 20%, 30%, 40%, 50%, 60%, 70%, or any other suitable percent of the plurality of cell-culture substrates).

The plurality of cell-culture substrates can include a plurality of pores (e.g., the cell-culture substrates can be formed, at least partially, from a mesh or mesh-like material). The porosity of the growth substrates may be different than the porosity of the spacer substrates. The plurality of cell-culture substrates can have a first area (e.g., a lateral area). The lateral area of the growth substrates (e.g., a first lateral area) may be different than the lateral area of the spacer substrates (e.g., a second lateral area). Such configurations may enhance the flow of nutrients and/or gasses around the plurality of cell-culture substrates such that cell or tissue growth is enhanced. A growth substrate may be disposed or located (e.g., along the shaft) at one or more predetermined distances from an adjacent growth substrate or spacer substrate (e.g., at least 1 μm, 2 μm, 5 μm, 10 μm, 100 μm, 1 cm, 5 cm, or any other suitable distance). Such a configuration may also enhance the flow of nutrients and/or gasses around the plurality of cell-culture substrates such that cell or tissue growth is enhanced. In any of the embodiments described herein, a first growth substrate of the plurality of growth substrates may have the same lateral area as a first spacer substrate of the plurality of spacer substrates. A first growth substrate of the plurality of growth substrates may have a smaller lateral area than a first spacer substrate of the plurality of spacer substrates. A first growth substrate of the plurality of growth substrates may have a larger lateral area than a lateral area of a first spacer substrate of the plurality of spacer substrates. A first growth substrate of the plurality of growth substrates may have the same lateral area as a second growth substrate of the plurality of growth substrates. A first growth substrate of the plurality of growth substrates may have a larger lateral area than a lateral area of a second growth substrate of the plurality of growth substrates. A first spacer substrate of the plurality of spacer substrates may have the same lateral area as a second spacer substrate of the plurality of spacer substrates. A first spacer substrate of the plurality of spacer substrates may have a larger lateral area than a lateral area of a second spacer substrate of the plurality of spacer substrates.

Tissue-engineering bioreactors (e.g., tissue-engineering bioreactor systems) and methods of use thereof can be utilized in the animal cell-based production of food products for human and animal consumption (e.g., cell-based meat and cell-based fat). Cell-based meats and cell-based fats generally comprise at least a preparation of adherent animal cells, including any one or combination of cells (e.g., muscle cells, connective tissue cells, fat cells, stem cells, mesenchymal cells, vascular cells, blood-associated cells, liver cells, nervous system cells, or cells from any edible animal organ, etc.). In some cell-based meats and cell-based fats, the preparations of adherent animal cells may be grown on an edible cell-culture substrate (e.g., an edible tissue-engineering scaffold) and can include at least a portion of the extracellular matrix that the cells have formed on the edible cell-culture substrate. In some cell-based meats and cell-based fats, the preparations of adherent animal cells may be processed by any one of a variety of methods (e.g., by centrifugation to reduce water content or compounding with other edible materials (e.g., texturizers, stabilizers, etc.)).

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Unless otherwise defined, all technical terms used herein have the same meaning as commonly understood by one of ordinary skill in the art to which this disclosure belongs or relates. As used in this specification and the appended claims, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than,” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than,” “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

As used herein, like characters refer to like elements.

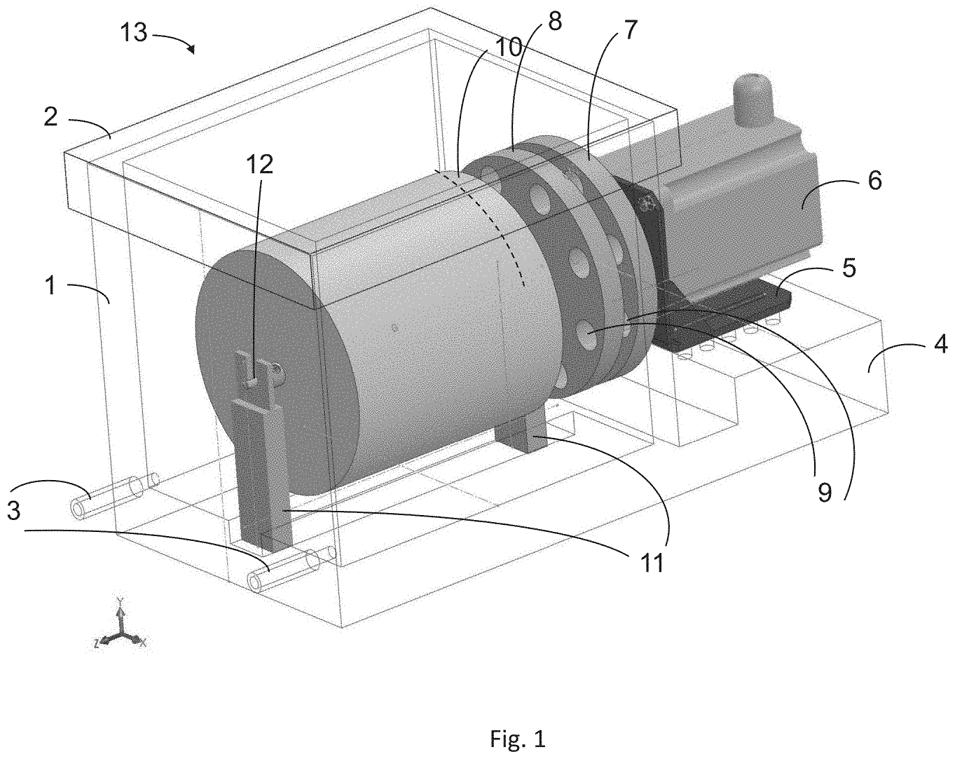

FIG. 1 shows a bioreactor 13 comprising a container 1; a lid 2 for the container 1; a plurality of openings 3 (e.g., for connecting the container 1 to tubing for culture media exchanges, for introducing a probe (e.g., a dissolved oxygen probe), etc.); a stand, stage, or feature 4 (e.g., an extension of the container and/or lid for affixing a motor and/or actuator bracket 5); a motor and/or actuator bracket 5 (e.g., for positioning and affixing a motor 6 relative to the container 1 and/or the lid 2); a motor and/or actuator 6 (e.g., a stepper motor); an external magnetic drive wheel 7 comprising one or more magnets arranged around and coupled to the drive shaft of the motor 6 (e.g., by way of a shaft collar); an internal magnetic drive wheel 8 comprising one or more magnets arranged around and coupled to an internal shaft 12, wherein the arrangement and polarity of the magnets on the internal magnetic drive wheel 8 are substantially aligned with and of opposite polarity as those of the external magnetic drive wheel 7, thereby resulting in attraction between the external and internal magnets and enabling non-contact linear and rotary force transmission from the outside to the inside of the container 1; one or more magnets 9 fitted into appropriately dimensioned holes in or otherwise affixed to the external and the internal magnetic drive wheels 7, 8; and a stack of one or more disks or substrates 10 (illustrated schematically in FIG. 1 as a solid cylinder representing a stack of individual circular disks) arranged on and affixed to the internal shaft 12 (e.g., by way of compression between shaft collars) which, in some embodiments comprise interleaved or alternating “growth” and “spacer” disks.

The stack of cell-culture substrates 10 may include alternating or interleaved growth disks or growth substrates and spacer disks or spacer substrates. Every other cell-culture substrate may be a growth substrate. Likewise, every other cell-culture substrate may be a spacer substrate. Other configurations of the growth substrates and spacer substrates are also within the scope of the present disclosure. For example, two growth substrates may be interleaved with one spacer substrate. In another example, two spacer substrates may be interleaved with one growth substrate. Any suitable number of growth substrates may be interleaved with any suitable number of spacer substrates or vice versa. The growth substrate may be different than the spacer substrate. Stated another way, the growth substrate may be formed from a different material than the spacer substrate. The diameter of the growth substrate may be greater than the diameter of the spacer substrate or vice versa. The growth substrates may include a greater number of pores than the spacer substrate or vice versa. The growth substrates may include pores with an average size that is greater than the average size of the pores of the spacer substrates or vice versa. Other characteristics as described herein may also be different between the growth substrates and the spacer substrates including the presence or absence of a coating, the shape, the angle relative to the shaft, the thickness, the cross-sectional shape of the structural elements of the substrates, (e.g., circular, square, triangular, etc.), presence or absence of surface texture on the structural elements of the substrates, presence or absence of electrical charge of the structural elements of the substrates, the type of weave in the case of woven substrates (e.g., plain, twill, etc.), etc.

The growth disks may comprise a porous lattice-like or otherwise porous structure capable of supporting high-density cell growth (e.g., a stainless steel wire mesh). The spacer disks may comprise a substantially more porous lattice-like or otherwise porous structure capable of allowing fluid and gas transport to and from the surfaces of the growth disks. In some embodiments, the stack of one or more disks or substrates 10 may comprise a tissue-engineering scaffold and supporting structures 11 to position and allow rotary and/or linear motion of the internal shaft 12, associated stack of disks 10, and internal magnetic drive wheel 8. In some embodiments, the supporting structures 11 may include bearings. In other embodiments, the internal shaft 12 may rest on or within a feature of the supporting structures 11. The internal shaft 12 may link the stack of disks 10 to the internal magnetic drive wheel 8, which in turn can be magnetically coupled to the external magnetic drive wheel 7, and thereby, in turn, may be linked to the drive shaft of the motor 6, enabling the motor 6 to turn the stack of disks 10. In the depicted embodiment of the bioreactor system 13, the shaft collars on either side of the stack of disks 10 can be utilized to position and affix the stack of disks 10 to the internal shaft 12 through, for example, compression and friction. In some embodiments, the shaft collars are replaced by any suitable element for the fixation of the disk or substrate stack 10 to the shaft 12.

With continued reference to FIG. 1, the container 1 and the lid 2 can provide a substantially sterile boundary within which can be received at least one solid, fluid, gas, and/or vapor component of the scalable bioreactor system. An example of a container 1 may be a rigid, or substantially rigid, and rectangular box with a length, width, and height. The container 1 may include walls having wall thickness(es) and an open side capable of being oriented substantially opposite to the direction of gravity. The open side of the container 1 may be capable of receiving a fluid (e.g., a cell-culture medium) and the container 1 may be configured to hold the fluid. The container 1, or at least a portion of the container 1, may be fabricated or formed from a material (e.g., a sterilizable material). The material may include at least one of Ultem™ polyetherimide, a polymer, a glass, a stainless steel, a corrosion-resistant metal, an alloy, a ceramic, a combination thereof, or any other suitable material. The ceramic may include at least one of Ultem™ polyetherimide, parylene, silicone, polytetrafluoroethylene (PTFE), polylactic acid (PLA), polysulfone, polyether ether ketone (PEEK), polypropylene, polycarbonate, borosilicate glass, a combination thereof, or any other suitable ceramic. Other shapes of the container 1 (e.g., square, circular, oval, a non-traditional shape, etc.) are also within the scope of the present disclosure.

An example of a lid 2 may be a rigid, or substantially rigid, rectangular box with a length, width, and height. The lid 2 may further include one or more walls, wherein the walls have thickness(es), and an open side capable of being oriented substantially toward the direction of gravity. An inside length and width of the lid 2 may enable the lid 2 to be fitted around and/or over the smaller outside length and width of the open side of the container 2. In certain embodiments, the lid 2 may include a partially overlapping height and loose-fit (e.g., substantially loose fit) over the open side of the container 2. The lid 2 (e.g., when coupled to the container 1) may enable aseptic exchange of gas with an environment external to the container 1 and the lid 2 (an example of which is the 5% CO₂ humidified air present within a standard CO₂ cell-culture incubator). The lid 2 may be fabricated or formed from one or more sterilizable materials as described in reference to the container 1. For example, the lid 2 may be formed from Ultem™ polyetherimide. Other shapes of the lid 2 (e.g., square, circular, oval, a non-traditional shape, etc.) are also within the scope of the present disclosure.

The container 1 and the lid 2 may comprise (e.g., may be formed from) any materials (e.g., materials of construction) or combinations of materials compatible with the desired use, scale, and configuration of a scalable bioreactor system a disclosed herein. Exemplary materials of construction of the container 1 and/or the lid 2 include, but are not limited to, polymers, glasses, stainless steel, corrosion-resistant metals, alloys, ceramics, any combination thereof, or any other suitable material. The ceramics may include Ultem™ polyetherimide, parylene, silicone, polytetrafluoroethylene (PTFE), polylactic acid (PLA), polysulfone, polyether ether ketone (PEEK), polypropylene, polycarbonate, borosilicate glass, any combination thereof, or any other suitable ceramic. The container 1 and the lid 2 may be fabricated by any method, combinations of methods, pre- and/or post-fabrication processing steps, and/or assembly steps compatible with the materials of construction, including, but not limited to, machining, welding, ultrasonic welding, casting, molding, thermoforming, 3D printing, fused deposition modeling, stereolithography, vapor polishing, conformal coating, etc.

In some embodiments, the container 1 and/or the lid 2 may be 3D printed from an FDA-compliant, food-contact approved, sterilizable, and 3D-printing compatible version of Ultem™ (e.g., Ultem™ 1010). In certain embodiments, the container 1 and/or the lid 2 may be conformal coated with an FDA-compliant, food-contact approved, sterilizable polymer (e.g., parylene). In various embodiments, the container 1 and/or the lid 2 may be conformal coated with an antimicrobial parylene-based polymer (e.g., SCS microRESIST). In some embodiments, the container 1 and/or the lid 2 may be fabricated from an FDA-compliant, food-contact approved, sterilizable, and corrosion-resistant grade of stainless steel (e.g., 316 stainless steel). In certain embodiments, the container 1 and/or the lid 2 may be passivated. In various embodiments, the container 1 and/or the lid 2 may be electropolished. In some embodiments, the container 1 and/or the lid 2 may be plated with metal (e.g., gold). In certain embodiments, the container 1 and/or the lid 2 may be solid but not rigid (e.g., not substantially rigid) comprising, for example, but not limited to, a polymeric bioprocess container and/or lid. In various embodiments, the container 1 and/or the lid 2 may be compostable (e.g., substantially compostable or at least partially compostable). In some embodiments, the container 1 and/or the lid 2 are not solid (e.g., not substantially solid) with one or more walls comprising, but not limited to, a laminar air sheet, a laminar liquid sheet, and/or a UV light sheet. In certain embodiments, the container 1 and/or the lid 2 may comprise an environmentally-controlled room. In various embodiments, the container 1 and/or the lid 2 may be disposed in an environmentally-controlled room. In some embodiments, the container 1 may comprise a fabricated and/or enclosed heated pool.

The container 1 and the lid 2 may have suitable dimensions that depend on the desired use, scale, and configuration of the scalable bioreactor system disclosed herein. The container 1 and the lid 2 may have mutually compatible dimensions that depend on the desired use, scale, and configuration of the scalable bioreactor system disclosed herein. An interior length of the container 1 may be 0.01 meters (m) to 100 m, 0.01 m to 75 m, 0.01 m to 50 m, 0.01 m to 25 m, 0.01 m to 10 m, 0.01 m to 1 m, 0.01 m to 0.5 m, 0.1 m to 100 m, 0.5 m to 100 m, 1 m to 100 m, 0.1 m to 50 m, 0.25 m to 25 m, 0.75 m to 10 m, 1 m to 5 m, 0.01 m to 0.1 m, 0.01 m to 0.2 m, 0.01 m to 0.3 m, 0.01 m to 0.5, or any other suitable interior length. An interior width of the container 1 may be 0.01 m to 100 m, 0.01 m to 75 m, 0.01 m to 50 m, 0.01 m to 25 m, 0.01 m to 10 m, 0.01 m to 1 m, 0.01 m to 0.5 m, 0.1 m to 100 m, 0.5 m to 100 m, 1 m to 100 m, 0.1 m to 50 m, 0.25 m to 25 m, 0.75 m to 10 m, 1 m to 5 m, 0.01 m to 0.1 m, 0.01 m to 0.2 m, 0.01 m to 0.3 m, 0.01 m to 0.5, or any other suitable interior width. An interior height of the container 1 may be 0.01 m to 100 m, 0.01 m to 75 m, 0.01 m to 50 m, 0.01 m to 25 m, 0.01 m to 10 m, 0.01 m to 1 m, 0.01 m to 0.5 m, 0.1 m to 100 m, 0.5 m to 100 m, 1 m to 100 m, 0.1 m to 50 m, 0.25 m to 25 m, 0.75 m to 10 m, 1 m to 5 m, 0.01 m to 0.1 m, 0.01 m to 0.2 m, 0.01 m to 0.3 m, 0.01 m to 0.5 m, or any other suitable interior height.

A wall thickness of the one or more walls of the container 1 may be 0.01 m to 50 m, 0.01 m to 25 m, 0.01 m to 15 m, 0.01 m to 5 m, 0.01 m to 1 m, 0.01 m to 0.5 m, 0.01 m to 0.1 m, 0.1 m to 1 m, 0.5 m to 1 meter, 0.1 m to 1 m, 0.2 m to 0.9 m, 0.3 m to 0.55 m, 0.01 m to 0.1 m, 0.01 m to 0.2 m, 0.01 m to 0.3 m, 0.01 m to 0.5 m, or any other suitable wall thickness. Each of the walls of the container 1 may have substantially the same thickness. Each of the walls of the container 1 may have different thicknesses. The walls of the container 1 may have any suitable combination of thicknesses (e.g., the first wall and the second wall may be a first thickness and the third wall and the fourth wall may have a second thickness, wherein the first thickness is different than the second thickness).

An exterior length of the container 1 may be 0.01 m to 100 m, 0.01 m to 75 m, 0.01 m to 50 m, 0.01 m to 25 m, 0.01 m to 10 m, 0.01 m to 1 m, 0.01 m to 0.5 m, 0.1 m to 100 m, 0.5 m to 100 m, 1 m to 100 m, 0.1 m to 50 m, 0.25 m to 25 m, 0.75 m to 10 m, 1 m to 5 m, 0.01 m to 0.1 m, 0.01 m to 0.2 m, 0.01 m to 0.3 m, 0.01 m to 0.5 m, or any other suitable interior length. An exterior width of the container 1 may be 0.01 m to 100 m, 0.01 m to 75 m, 0.01 m to 50 m, 0.01 m to 25 m, 0.01 m to 10 m, 0.01 m to 1 m, 0.01 m to 0.5 m, 0.1 m to 100 m, 0.5 m to 100 m, 1 m to 100 m, 0.1 m to 50 m, 0.25 m to 25 m, 0.75 m to 10 m, 1 m to 5 m, 0.01 m to 0.1 m, 0.01 m to 0.2 m, 0.01 m to 0.3 m, 0.01 m to 0.5 m, or any other suitable interior width. An exterior height of the container 1 may be 0.01 m to 100 m, 0.01 m to 75 m, 0.01 m to 50 m, 0.01 m to 25 m, 0.01 m to 10 m, 0.01 m to 1 m, 0.01 m to 0.5 m, 0.1 m to 100 m, 0.5 m to 100 m, 1 m to 100 m, 0.1 m to 50 m, 0.25 m to 25 m, 0.75 m to 10 m, 1 m to 5 m, 0.01 m to 0.1 m, 0.01 m to 0.2 m, 0.01 m to 0.3 m, 0.01 m to 0.5, or any other suitable exterior height.

In some embodiments, the container 1 and/or the lid 2 may have any suitable shape or combination of shapes. A shape of the container 1 and/or the lid 2 may be rectangular, circular, triangular, cylindrical, square, amorphous (e.g., a non-traditional shape), fractal, or any other suitable shape. In various embodiments, the lid 2 may be configured to fit tightly (e.g., substantially tightly) to the open side of the container 1. The lid 2 may couple (or be couplable) to the container 1 by way of a gasket and screws, a snap fit, or any other suitable coupling mechanism. The lid 2 may comprise an integral feature of the container 1, wherein, for example, solid, fluid, gas, and/or vapor components of the scalable bioreactor system can be exchanged across the boundary (e.g., the substantially sterile boundary) of the container 1 and/or the lid 2 via one or more openings 3 in the container 1, the lid 2, or both the container 1 and the lid 2. In some embodiments, the container 1 and/or the lid 2 may include one or more stands, stages, or features 4. The stand 4 may include handles to facilitate positioning of the container 1 and/or the lid 2, an extension from the container 1 to affix or couple a motor or actuator bracket 5 to the container 1, etc.

The container 1, the lid 2, or both the container 1 and the lid 2 may incorporate one or more openings 3. In some embodiments, one or more of the openings 3 may serve one or more purposes, including, but not limited to: (a) the periodic, intermittent, semi-continuous, or continuous exchange of solid, liquid, gas, or vapor between the inside and outside of the substantially sterile boundary provided by the container 1 and the lid 2 and (b) the introduction of process monitoring or control elements, including temperature probes, dissolved gas probes (e.g., for dissolved oxygen or CO₂ measurement), pH probes, metabolite probes, conductivity probes, sampling ports (e.g., for off-line monitoring of media components or cell numbers, etc.), cell-seeding ports, cell-harvesting ports, etc. In some embodiments, an opening 3 may comprise a sight glass (e.g., for observation of the internal components or operation of the container 1).

In some embodiments, an opening 3 may comprise a glass, polymer, or otherwise substantially optically transparent material covering or lens for non-contact measurement of oxygen, pH, optical density (e.g., for cell number), etc. In certain embodiments, an opening may be utilized to introduce an electrical wire, fiber optic cable, tubing, or other conduit for any purpose between the inside and outside of the container 1 or the lid 2. In various embodiments, the opening may be utilized to introduce a shaft or other form of motion-control element between the outside and inside of the container 1 or the lid 2. In some embodiments, the container 1 may include at least two openings, one for use as a fresh culture-media inlet and one for use as a waste culture-media outlet. In certain embodiments, the lid 2 may include one or more openings.

In certain embodiments, openings may comprise 3D-printed hollow cylinders or barbed or otherwise stepped or textured cylinders that are integral to and extend from a 3D-printed container or lid and having inside and outside diameters and length consistent with serving as tubing fittings. In various embodiments, openings may comprise a hole through the wall of the container 1 or the lid 2 through which a bulkhead fitting is installed. In some embodiments, openings may comprise a hole through the wall of the container 1 or the lid 2 to which tubing fitting or sanitary fittings (e.g., Tri-clover or similar fittings) can be affixed to the wall around the hole by way of adhesive, welding, press-fit, or any other method or combination of methods of fixation. In certain embodiments, the opening may comprise a secondary lid or port for use in the manual or automated introduction of materials or parts into the inside of the container 1 or for making adjustments or repairs or replacement of parts within the container 1. In various embodiments, the one or more openings may be reversibly or irreversibly capped, plugged, closed via a valve, or otherwise rendered closed either temporarily or permanently for any reason. In some embodiments, openings may be utilized for seeding cells onto one or more cell-culture substrates. In certain embodiments, openings may further comprise nozzles and be utilized to seed cells by spraying cells onto one or more cell-culture substrates.

In some embodiments, one or more motors and/or actuators 6 may be brought into proximity of the container 1 and/or the lid 2, for example, for the purpose of positioning and/or moving periodically and/or continuously in any direction of linear and/or rotary motion any component internal and/or external to the container 1 and/or the lid 2. In some embodiments, a motor 6 (e.g., a stepper motor) affixed or coupled to a bracket 5, in turn affixed or coupled to a feature 4 of the outside of the container 1, may be utilized to rotate a shaft 12, shaft collars or other fixtures 13, and/or an associated stack of one or more cell-culture disks, substrates, or tissue-engineering scaffolds 10, by way of a magnetic coupling formed by way of an external magnetic wheel 7 and an internal magnetic wheel 8. In some embodiments, the motor and/or actuator 6 may not be physically connected to the container 1 and/or the lid 2 (i.e., not physically affixed or coupled to the feature 4 of the container 1 and/or the lid 2), instead being brought into proximity to the container 1 and/or the lid 2 by disposing or positioning the motor and/or actuator 6 in alignment with one or more mating components within or external to the container 1 and/or the lid 2.

In some instances, rotary motion may be transferred from the motor 6 to the external magnetic wheel 7 by way of a flexible shaft extension (e.g., for the purpose of situating the motor 6 outside of a temperature-controlled incubator and thereby substantially mitigating the transfer of motor-generated heat to the inside of the incubator). In certain embodiments, heat generated by the one or more motors may be utilized to heat an incubator. In various embodiments, heat generated by the one or more motors 6 may be utilized to heat a container (e.g., the container 1). In certain embodiments, a motor cooling system (e.g., based on a Peltier thermoelectric cooler, a coolant recirculation loop, a heat sink, a fan, or a combination thereof) may be utilized to cool the motor 6.

In various embodiments, a plurality of motors or actuators may be incorporated, enabling not only rotary motion about a substantially longitudinally oriented central axis but also multi-axis linear motion or rotary motion, for any one of a number of purposes, including, but not limited to: (a) facilitating cell seeding (e.g., by manipulating the angular orientation or disk-to-disk separation of the stack of cell-culture disks, substrates or tissue-engineering scaffolds, etc.) and (b) cell harvest (e.g., by repetitively shaking back and forth or tapping the stack of cell-culture disks, substrates, or tissue-engineering scaffolds, etc.). In some embodiments, the motor 6 may take the form of a shaker device (e.g., a shaker platform typically utilized in conjunction with shaker flasks, etc.), and the shaker device may be utilized to facilitate cell attachment, proliferation, differentiation on, or cell harvesting from the one or more cell-culture substrates within the container. In some embodiments, the shaker device may be utilized to stimulate engineered tissue formation by oxygenation, provision of fluid shear stress, or any other suitable method. In some embodiments, the motor 6 may take the form of a rocking device (e.g., a rocker platform typically utilized in conjunction with mixing fluids, etc.) and the rocking device may be utilized to facilitate cell attachment, proliferation, differentiation on, or cell harvesting from the one or more cell-culture substrates within the container. In some embodiments, the rocking device may be utilized to stimulate engineered tissue formation. The rocking device may stimulate engineered tissue formation by oxygenation, provision of fluid shear stress, etc. In some embodiments, one or more motors may be utilized to move a component of the system through an electromagnetic field. The component of the system may be moved through the electromagnetic field to induce an electric current in a component of the system (e.g., in the wires comprising a metal mesh disk), apply an electromagnetic field to the growing cells or tissues, etc. The plurality of motors or actuators may be used to adjust a position and/or an orientation of one or more components of the system based on one or more sensor readings. The sensor readings may be obtained using any of the sensors described herein, and may be associated with a performance or an operation of the bioreactors described herein.

In certain cases, a magnetic coupling comprising an external magnetic wheel 7, an internal magnetic wheel 8, and magnets 9 may be utilized to mediate the non-contact transmission of rotary motion from outside the container 1 (e.g., via a motor 6) to the inside of the container 1 (e.g., a shaft 12). In certain embodiments, the magnetic coupling may comprise a commercially available non-contact magnetic shaft coupling (e.g., from McMaster-Carr®). In various embodiments, the external magnetic wheel 7 and the internal magnetic wheel 8 may be designed (e.g., custom-designed) to incorporate a plurality of magnets disposed in a pattern. The pattern of magnets 9 may be amenable to magnetic force (e.g., robust magnetic force) and associated torque transmission across the wall of the container 1. The pattern may comprise a circular configuration, a linear configuration, or any other random or non-random spatial distribution that provides sufficient magnetic force and associated torque transmission across the wall of the container. The positions and orientations of the magnets may be manually or automatically adjusted based on a quantity or type of material or cell to be grown or cultivated, or based on one or more sensor readings associated with a performance or an operation of the bioreactor. In some embodiments, the external magnetic wheel 7 and the internal magnetic wheel 8 may, in lieu of having a circular cross-section, comprise substantially rectangular bars with magnets affixed at opposite ends of the external and internal rectangular bars in a pattern such as to be capable of transmitting magnetic force and torque across the wall of the container 1. The external magnetic wheel 7 and the internal magnetic wheel 8 may take any suitable shape, and any suitable pattern of magnets may be utilized to provide magnetic force and associated torque transmission across the wall of the container 1. In some embodiments, the materials of construction of the magnets 9 may be selected from any combination of magnetic materials and coatings consistent with their use, including but not limited to, neodymium, neodymium iron boron, and samarium cobalt to name a few magnet materials and polytetrafluoroethylene (PTFE), parylene, and gold, to name a few magnet coating materials. In some embodiments, one or more of the plurality of magnets may be electromagnets.

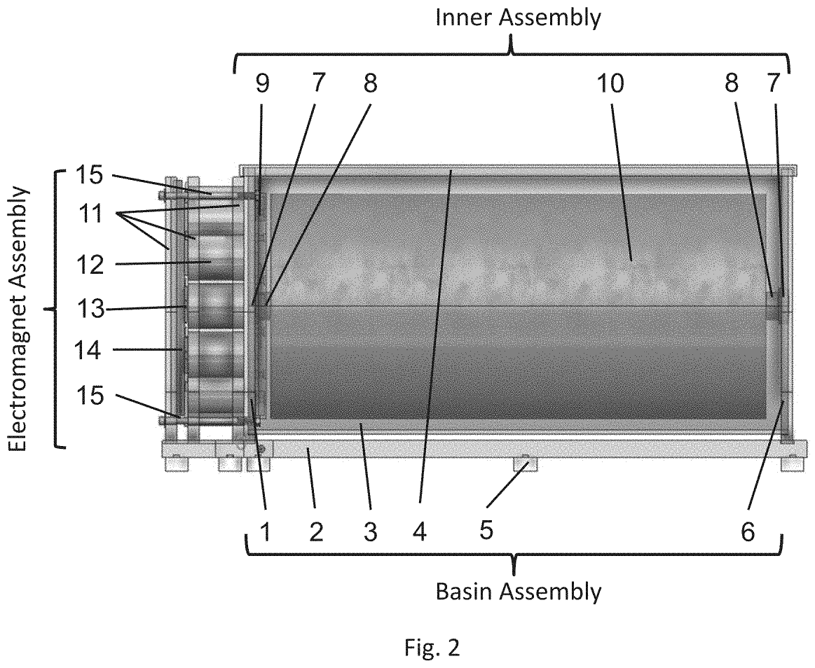

FIG. 2 shows an embodiment of a bioreactor in which disk stack rotation can be driven via electromagnets. The disk stack rotation may be controlled and/or adjusted manually or automatically based on a quantity or type of material or cells to be grown or cultivated, or based on one or more sensor readings associated with a performance or an operation of the bioreactor. The bioreactor can include a basin assembly, an inner assembly, and an electromagnet assembly. The basin assembly may include a front plate 1, a back plate 6, a basin base plate 2, a lid 4, side walls 3, and feet 5. The inner assembly may include a shaft (not shown), two shaft collars 8, two ball bearings 7, a stack of cell-culture substrates 10, and an internal magnetic drive wheel 9. The electromagnet assembly may include structural plates 11, couplers for assembly (for example, four bolts 15), a plurality of electromagnets (for example, twelve electromagnets 12). In some embodiments, the system may further comprise a cooling system, including, for example, one or more cooling elements (for example, twelve Peltier devices 13), one or more heat sinks (for example, aluminum water cooling blocks 14), and one or more additional liquid cooling system components (for example, pumps, fans, radiators, tubing, etc.). As discussed in reference to the bioreactor of FIG. 1, the stack of cell-culture substrates 10 may include alternating or interleaved growth disks or growth substrates and spacer disks or spacer substrates.

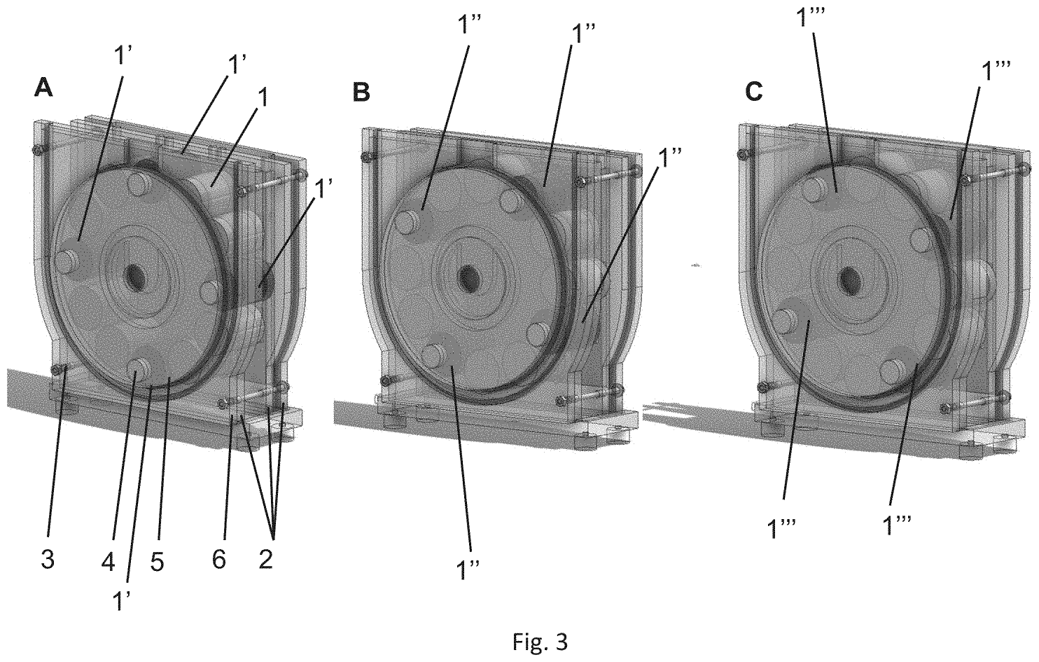

FIG. 3 illustrates an example of using electromagnets to drive disk stack rotation in the scalable bioreactor described in FIG. 2. The electromagnet assembly detailed in FIG. 2 is shown. Twelve electromagnets 1 are arranged along the perimeter of a circle. Structural plates 2 and through bolts 3 provide support for the electromagnets 1 and electromagnet cooling components (for example, the Peltier devices and aluminum cooling blocks). Only partial basin and inner assemblies detailed in FIG. 2 are shown. The inner magnetic wheel 5 includes a plurality of magnets 1 (for example, four circular magnetic disks 4). In this example, electric current is first applied to the four highlighted electromagnets 1′ in panel A of FIG. 3. The current creates a magnetic field and the inner magnetic disks 4 are attracted to the source of this field. Subsequently, the electric current is applied to the four highlighted electromagnets 1″ in panel B of FIG. 3 and then to the four highlighted electromagnets 1′″ in panel C of FIG. 3, whereby the resultant magnetic fields drive the inner magnetic disks 4 to rotate (for example, approximately 30 degrees clockwise) in each of these steps. The process described herein may be repeated multiple times to complete a full rotation (or a substantially full rotation). In the process described herein, the angular rotation of each step can be increased or reduced, for example, by progressively modulating or stepping down the current applied to one set of four electromagnets while modulating or stepping up the current applied to the next set of four electromagnets. The current applied to the electromagnets may be manually or automatically modulated based on a quantity or type of material or cells to be grown or cultivated, or based on one or more sensor readings associated with a performance or an operation of the bioreactor.

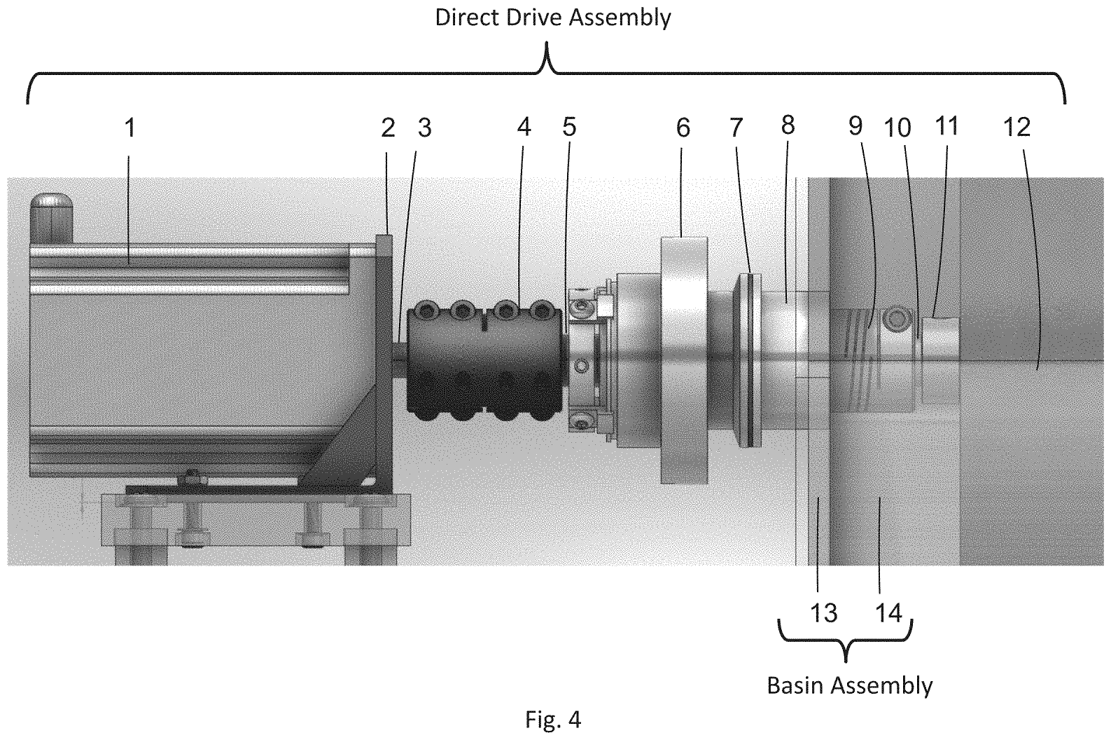

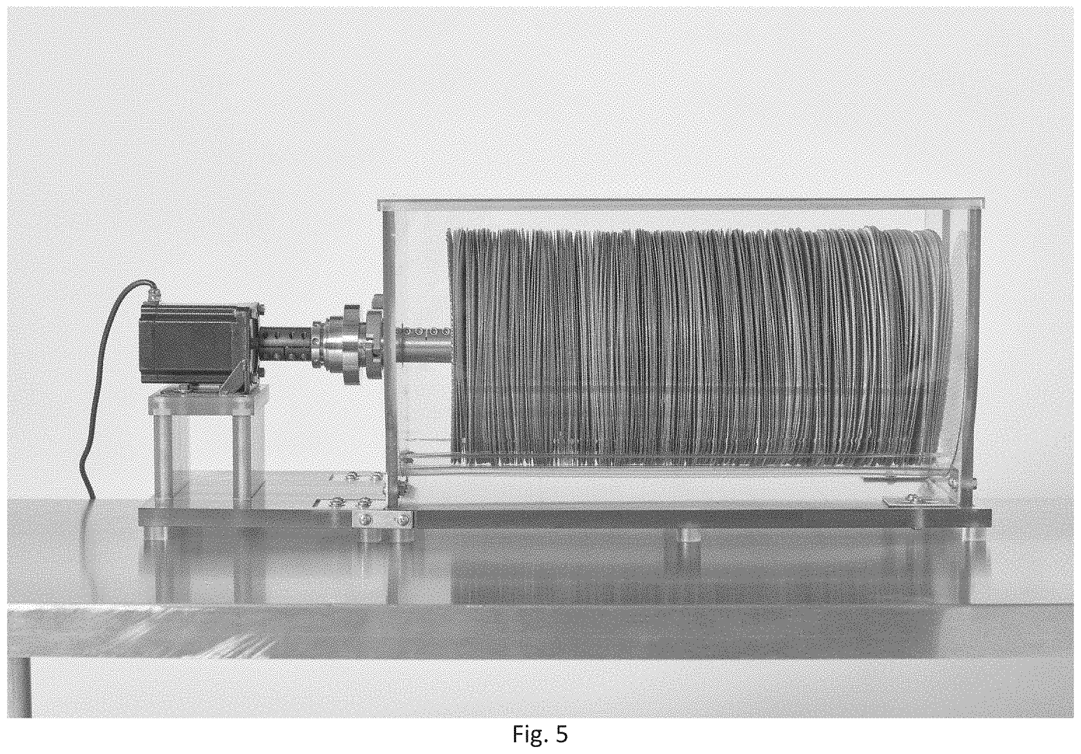

FIG. 4 depicts another embodiment of a scalable bioreactor system. The bioreactor comprises a basin assembly and a direct drive assembly. The figure only shows a partial basin assembly: the front plate 13 and the side wall 14. In this embodiment, the direct drive assembly comprises: a motor 1, a motor bracket 2, a motor shaft 3, an outer shaft coupler 4, an outer shaft 5, a mechanical seal 6, a gasket 7, a ferrule 8, a tri-clamp (not shown), an inner shaft coupler 9, an inner shaft 10, a shaft collar 11, and a disk stack 12. FIG. 5 shows a photo of a scalable bioreactor in which disk stack rotation is driven via a directly coupled motor as illustrated in FIG. 4.

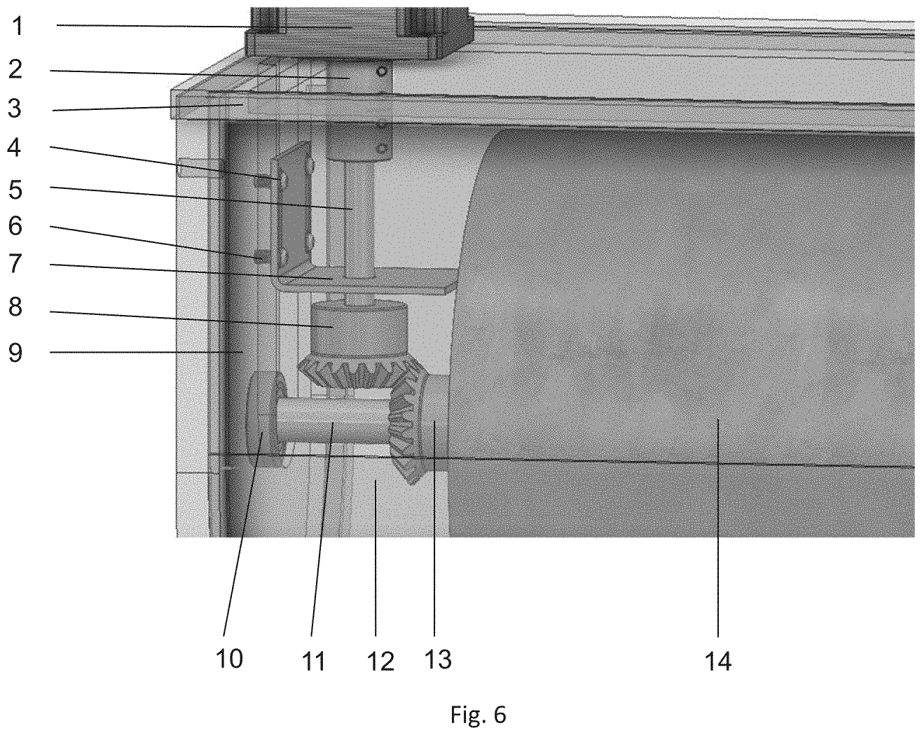

FIG. 6 shows a portion of a scalable bioreactor in which disk stack rotation is driven via a motor located on the top of the bioreactor container through the use of beveled gears. The bioreactor comprises a basin assembly and a bevel gear drive assembly. The figure only shows a partial basin assembly: a front plate 9, a lid 3, and a side wall 12. In this embodiment, the bevel gear drive assembly comprises: a motor 1, a shaft coupler 2, two shafts 5 and 11, two bevel gears 8 and 13, a structural bracket 7, threaded inserts 6, screws 4, a ball bearing 10, and a stack of cell culture substrates 14. In other embodiments, direct drives may comprise any combination of bevel gears, worm gears, flexible shafts, and/or other motion transmission elements.

Referring again to FIG. 1, in some instances, one or more cell-culture substrates (e.g., a stack of stainless steel wire mesh disks) or tissue-engineering scaffolds 10 may be configured or disposed on a shaft 12 or otherwise configured in any way as to enable any combination of rotary, angular, or linear motion to be transferred to the stack of cell-culture substrates 10. The shaft 12 may be configured on one or more shaft supports 11 or otherwise configured so as to enable any combination of rotary, angular, or linear motion of the shaft (e.g., via bearing mounts, etc.). In some embodiments, a length of the shaft 12 may be 0.01 meters (m) to 100 m. For example, the length of the shaft 12 may be 0.01 m to 90 m, 0.01 m to 75 m, 0.01 m to 50 m, 0.01 m to 25 m, 0.01 m to 10 m, 0.01 m to 1 m, 0.1 m to 90 m, 0.1 m to 75 m, 0.1 m to 50 m, 0.1 m to 25 m, 0.1 m to 10 m, 0.1 m to 1 m, 0.5 m to 90 m, 0.5 m to 75 m, 0.5 m to 50 m, 0.5 m to 25 m, 0.5 m to 10 m, 0.5 m to 1 m, 1 m to 90 m, 1 m to 75 m, 1 m to 50 m, 1 m to 25 m, 1 m to 10 m, 1 m to 5 m, or any other suitable length.

The diameter of the shaft 12 or the diameter of at least a portion of the shaft 12 may be 200 microns to 10 m. For example, the diameter of the shaft 12 or the diameter of at least a portion of the shaft 12 may be 200 microns to 10 m, 200 microns to 5 m, 200 microns to 1 m, 200 microns to 0.5 m, 200 microns to 0.1 m, 1000 microns to 10 m, 1000 microns to 5 m, 1000 microns to 1 m, 1000 microns to 0.5 m, 1000 microns to 0.1 m, 0.1 m to 10 m, 0.1 m to 5 m, 0.1 m to 1 m, 0.1 m to 0.5 m, or any other suitable diameter. In certain embodiments, the shaft 12, the shaft supports 11, or other elements of the bioreactor 13 (e.g., the container 1) may be configured to vibrate (e.g., to facilitate cell harvesting, to deter cell attachment, etc.). In various embodiments, the shaft 12 may be hollow or perforated (e.g., to further serve as a tube or distributor of fluid, to promote gas or nutrient transport, to facilitate cell harvesting, etc.). In some embodiments, the shaft 12 may be configured to rotate from a substantially horizontal to a substantially vertical configuration (e.g., to facilitate seeding of the cell-culture substrates, etc.). The shaft may be configured to rotate about any axis in three-dimensional space. The body of the shaft may be moved relative to one or more components of the bioreactor using one or more motors or actuators. In certain cases, the shaft 12 may be solid or semi-solid (i.e., hollow or partially hollow).

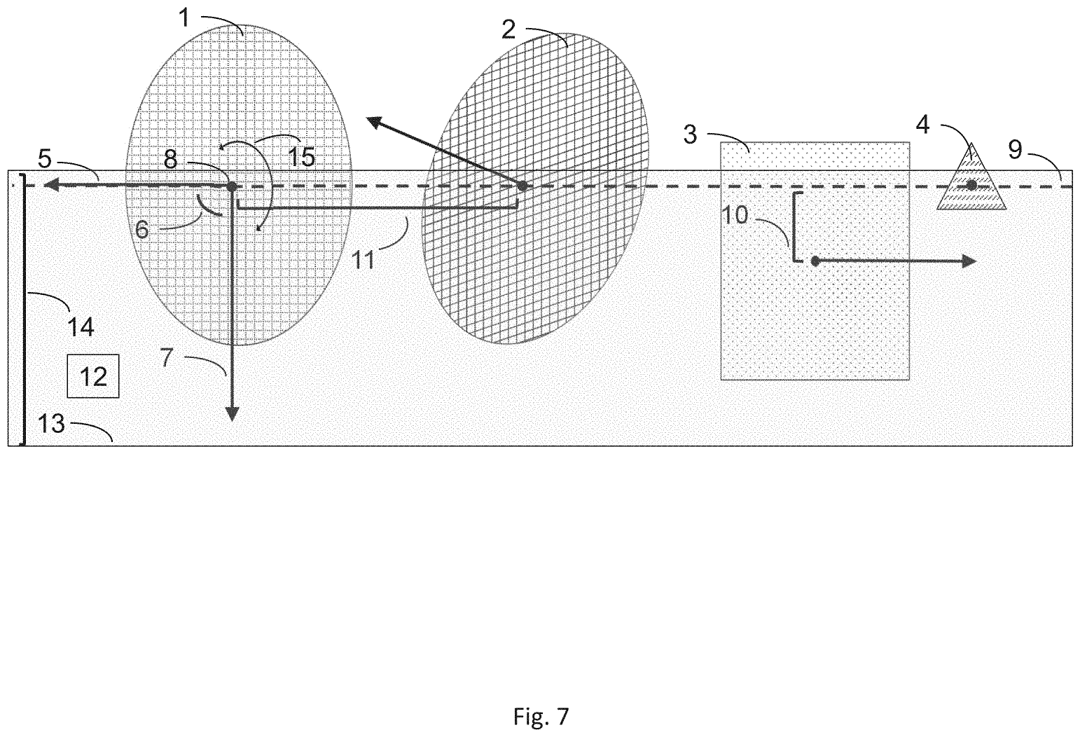

FIG. 7 shows embodiments of cell-culture substrates. A first cell-culture substrate 1 having a suitable planar shape (e.g., circular, rectangular, triangular, etc.), planar area, geometric center of the planar area (i.e., centroid) 8, and thickness can be spatially or temporally configured relative to a vector substantially normal to the local force of gravity, artificial gravity, or net centrifugal force 7 and one or more lines 9 (e.g., an axis of rotation) by way of a vector substantially normal to the planar area of the first cell-culture substrate 5, angles of inclination or declination 6 between 5 and 7 or between 5 and any other vector, and distances 10 between the centroid of the planar area 8 and any one of one or more lines 9 (e.g., offsets between the centroid 8 and an axis of rotation).

The spatial or temporal configuration of a second cell-culture substrate 2, a third cell-culture substrate 3, or any suitable number of cell-culture substrates (e.g., as depicted schematically by a fourth or last cell-culture substrate 4) can be defined by their respective normal vectors and centroids relative to the vector substantially normal to the planar area of the first cell-culture substrate 5 and the centroid of the planar area of the first cell-culture substrate 8, including by way of distances 11 between the centroids of any cell-culture substrates (e.g., as in a gap distance between cell-culture substrates). In certain embodiments, a container and lid enclosing one or more cell-culture substrates may be partially filled with one or more of a solid, fluid, gas, or vapor media 12. The height of the media 14 may be defined relative to the inside bottom or inside surface 13 of the container. The media height 14 can be alternatively defined relative to one or more lines 9 or relative to the centroids of the planar areas of the one or more cell-culture substrates 8, thereby defining the distances between the centroids of any one or more of a plurality of cell-culture substrates relative to the surface of the media.

A cell-culture substrate can be understood by virtue of its planar area and thickness (i.e., volume) to possess a set of substantive material and/or surface properties including, but not limited to, a density, a porosity, a stiffness, a strength, an elasticity, a surface energy, a surface roughness, etc. The material and/or surface properties may influence the mass transport properties, cell-culture properties, or any other properties of the cell-culture substrate. In some embodiments, one or more of the plurality of cell-culture substrates may be substantially curved, undulated, or corrugated (e.g., to facilitate gas transport, fluid drainage, etc.). In certain embodiments, one or more of the plurality of cell-culture substrates may have spring-like properties (e.g., to facilitate control of the tightness of the disk stack, etc.). In various embodiments, a first cell-culture substrate 1 having a suitable planar shape (e.g., circular, rectangular, triangular, etc.), planar area, geometric center of the planar area (i.e., centroid) 8, and thickness can be spatially or temporally configured relative to a vector substantially normal to the local force of gravity, artificial gravity, or net centrifugal force and one or more lines 9 (e.g., an axis of rotation) by way of a vector substantially normal to the planar area of the first cell-culture substrate 5, angles of inclination or declination 6 between 5 and 7 or between 5 and any other vector, and distances 10 between the centroid of the planar area 8 and any one of one or more lines 9 (e.g., offsets between the centroid and an axis of rotation).

A thickness of the growth substrates may be 100 microns to 10,000 microns, 100 microns to 1,000 microns, 100 microns to 900 microns, 100 microns to 800 microns, 100 microns to 700 microns, 100 microns to 600 microns, 100 microns to 500 microns, 100 microns to 400 microns, 100 microns to 300 microns, 100 microns to 200 microns, 200 microns to 10,000 microns, 200 microns to 9,000 microns, 200 microns to 8,000 microns, 200 microns to 7,000 microns, 200 microns to 6,000 microns, 200 microns to 5,000 microns, 200 microns to 4,000 microns, 200 microns to 3,000 microns, 200 microns to 2,000 microns, 200 microns to 1,000 microns, 200 microns to 500 microns, or any other suitable thickness. A thickness of the spacer substrates may be 100 microns to 10,000 microns, 100 microns to 1,000 microns, 100 microns to 900 microns, 100 microns to 800 microns, 100 microns to 700 microns, 100 microns to 600 microns, 100 microns to 500 microns, 100 microns to 400 microns, 100 microns to 300 microns, 100 microns to 200 microns, 200 microns to 10,000 microns, 200 microns to 9,000 microns, 200 microns to 8,000 microns, 200 microns to 7,000 microns, 200 microns to 6,000 microns, 200 microns to 5,000 microns, 200 microns to 4,000 microns, 200 microns to 3,000 microns, 200 microns to 2,000 microns, 200 microns to 1,000 microns, 200 microns to 500 microns, or any other suitable thickness.

The spatial or temporal configuration of a second cell-culture substrate 2, a third cell-culture substrate 3, or any suitable number of cell-culture substrates (e.g., as depicted schematically by a fourth or last cell-culture substrate 4) can be defined by their respective normal vectors and centroids relative to the vector substantially normal to the planar area of the first cell-culture substrate 5 and the centroid of the planar area of the first cell-culture substrate 8, including by way of distances 11 between the centroids of any cell-culture substrates (e.g., as in a gap distance between cell-culture substrates).

In some cases, a gap or gap distance between the cell-culture substrates may be 0 cm to 100 cm. For example, the gap distance between cell-culture substrates may be 0.1 cm to 100 cm, 0.1 cm to 75 cm, 0.1 cm to 50 cm, 0.1 cm to 10 cm, 0.1 cm to 1 cm, 0.1 cm to 0.5 cm, 0.5 cm to 100 cm, 0.5 cm to 75 cm, 0.5 cm to 50 cm, 0.5 cm to 10 cm, 0.5 cm to 1 cm, 0.5 cm to 0.75 cm, 1 cm to 100 cm, 1 cm to 75 cm, 1 cm to 50 cm, 1 cm to 10 cm, 1 cm to 5 cm, 1 cm to 1.5 cm, or any other suitable gap distance. In certain cases, a container and lid enclosing one or more cell-culture substrates may be partially filled with one or more of a solid, fluid, gas, or vapor media 12. The height of the media 14 may be defined relative to the inside bottom of the container 13. In certain embodiments, and at any temporal point in the cell-growth process in the bioreactor, the media height relative to the inside height of the container may range from zero (0) percent to one hundred (100) percent of the inside height of the container. For example, the media height relative to the inside height of the container may be 1% to 90%, 1% to 75%, 1% to 50%, 1% to 25%, 1% to 25%, 1% to 10%, 1% to 5%, 10% to 90%, 15% to 85%, 20% to 80%, 25% to 75%, 30% to 70%, 35% to 65%, 40% to 60%, 45% to 55%, or any other suitable percentage of the inside height of the container. The media height 14 can therefore be alternatively defined relative to one or more lines 9 or relative to the centroids of the planar areas of one or more cell-culture substrates 8 thereby defining the distances between the centroids of any one or more cell-culture substrates relative to the surface of the media. A cell-culture substrate may be understood by virtue of its planar area and thickness (i.e., volume) to possess a set of substantive material and surface properties including, but not limited to, a density, a porosity, a stiffness, a strength, an elasticity, a surface energy, a surface roughness, biodegradation characteristics, suitability of use in food applications (i.e., edibility), etc. Any number of the material and/or surface properties may be understood to influence the mass transport properties, cell-culture properties, or any other properties of the cell-culture substrate.

Further to the exemplary material properties, in some embodiments, the cell-culture substrates may comprise tissue-engineering scaffolds for use in forming any type of tissue, including, but not limited to, cell-based meat and cell-based fat. In some embodiments, the cell-culture substrate may comprise any one of a number of substantially non-degradable materials, including, but not limited to, meshes or textiles formed from any cleanable and sterilizable polymers, metals, glasses, ceramics, etc. Examples of the materials include, but are not limited to, polyethylene terephthalate (PET) polyester, polyether ether ketone (PEEK), polypropylene, polyetherimide (Ultem™); any substantially corrosion-resistant metal, including, but not limited to, stainless steel (e.g., 304 stainless steel and 316 stainless steel), titanium, etc.; borosilicate glass, titanium dioxide ceramic, etc. In some embodiments, the substantially non-degradable cell-culture substrate may comprise a mesh-like lattice including, for example, but not limited to, a lattice including 3-D printed Ultem™ 1010. In some embodiments the cell-culture substrate may comprise any one of a number of substantially degradable materials, including, but not limited to, poly(glycolic acid) (PGA), poly(L-lactic acid) (PLLA), poly(lactide-co-glycolide) (PLGA), polycaprolactone (PCL), polyglycerol sebacate (PGS), etc.

In some instances, the edible cell-culture substrate may comprise any one of a number of edible materials, including sugar-based, carbohydrate-based, protein-based, or fat-based edible materials. Such edible materials may include, but are not limited to, pectin, chitosan, grain products (including, rice-based products, corn-based products, wheat-based products, seaweed-based products, algae-based products, etc.). In certain cases, the cell-culture substrates may comprise a corrosion-resistant wire mesh such as, for example, a stainless steel wire mesh. In various embodiments, the cell-culture substrates may comprise a textile, which may be substantially non-degradable. In some embodiments, the cell-culture substrates may comprise a textile, which may be substantially degradable. In certain embodiments, the cell-culture substrates may comprise a textile, which may be edible.

Referring again to FIG. 1, in some embodiments one or more motors 6 (e.g., a stepper motor) affixed to features 4 of the container 1 (e.g., via brackets 5) may be utilized to apply any combination of rotary, angular, or linear motion to the cell-culture substrates 10 inside the container 1. In certain embodiments, a rotary motion is provided at speeds ranging from 0 to 1000 revolutions per minute (RPM), durations of rotary motion ranging from 1 second to 1 year, and angular distances per instance of intermittent rotary motion ranging from 0 to 360 degrees. Furthermore, continuous angular rotation may be applied to one or more of the cell-culture substrates, and may be applied in any mode of motion or combination of modes of motion, including, but not limited to, continuously, periodically, intermittently, in alternating directions, in suitable motion patterns, at discretely or continuously changing rotational speeds, etc.

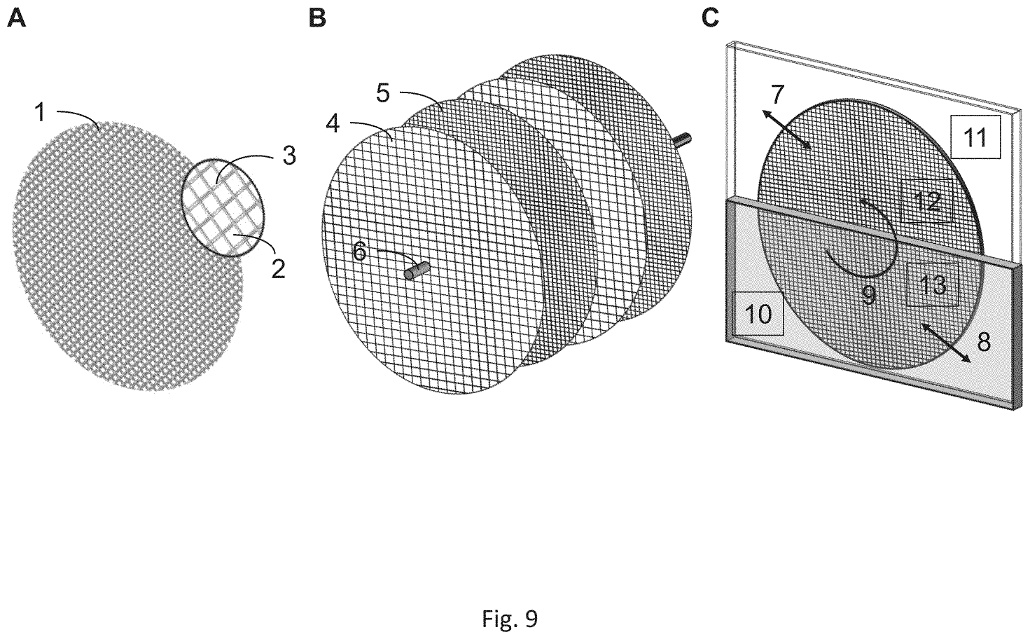

Referring to panel C of FIG. 9, in some embodiments, during rotary motion 9 of the plurality of one or more cell-culture substrates, a first portion 12 of the surface area of the cell-culture substrates is outside of the fluid media 10 partially filling the container and in the gas headspace 11 above the fluid media, while a second portion 13 of the surface area of the cell-culture substrates remains submerged in the fluid media 10. The surface area of the first portion 12 can vary at any stage of the scalable bioreactor process. At some stages in the process (e.g., during cell attachment) the surface area of the first portion 12 can be zero, for example, wherein the fluid media covers the entirety of the plurality of one or more cell-culture substrates during the cell attachment process.

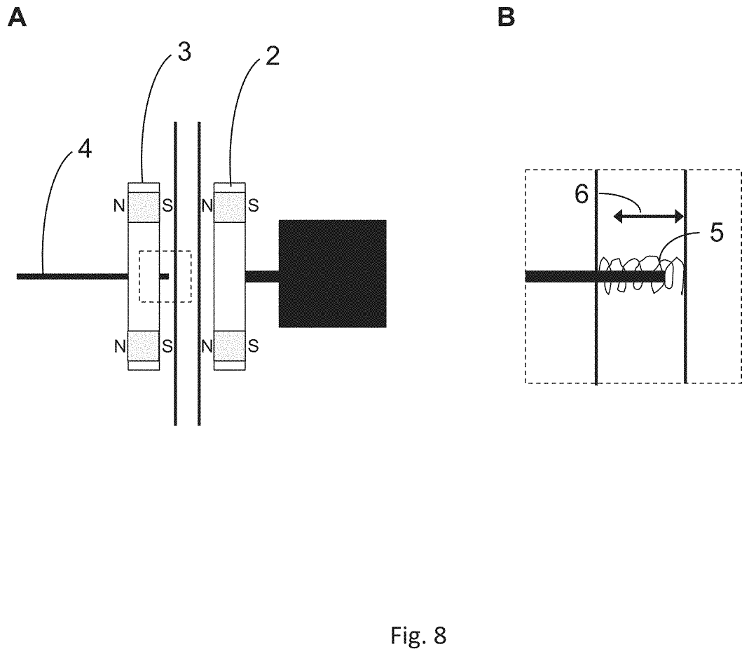

Referring to FIG. 8, in certain embodiments, the distance-dependent forces of attraction or repulsion between the magnets of the external magnetic wheel and internal magnet wheel may be utilized to pull or push, including, for example, but not limited to, retaining the internal magnetic wheel in close proximity to the inside wall of the container and repeatedly and/or rapidly tapping the shaft and associated stack of disks against the inside wall of the container (e.g., in conjunction with a spring or spring-like device and second motor or actuator), etc.

Panel A of FIG. 8 shows an example of a first method for mimicking manual tapping utilized in harvesting cells from the surface of a tissue culture flask. The first method can facilitate cell harvesting. The method may comprise repeatedly and/or rapidly bringing a motor 1 and external magnetic wheel 2 closer and then farther from the wall of the container and the internal magnetic wheel 3 coupled or connected to a shaft 4 (e.g., by way of a second motor, linear stage, actuator, solenoid, or other automated, semi-automated, or manual component). The movement of the motor and external magnetic wheel relative to the internal magnetic wheel can be used to modulate an electromagnetic field that drives a motion or a movement of the shaft and any cell-culture substrates coupled to or associated with the shaft. The movement of the motor and external magnetic wheel may be controlled manually by an operator, or automatically based on a quantity or type of material or cell to be grown or cultivated, or based on one or more sensor readings associated with a performance or an operation of the bioreactor. In some cases, the movement of the motor and external magnetic wheel may be controlled manually or automatically at one or more user-defined or user-selected timepoints, or at one or more predetermined timepoints corresponding to one or more time periods of interest during a cell growth/harvesting procedure.

Panel B of FIG. 8 shows an example of a second method for mimicking manual tapping utilized in harvesting cells from the surface of a tissue culture flask and thereby, for example, facilitating cell harvesting. The method may comprise utilizing a spring 5 or spring-like device to retain the end of the shaft at a substantially fixed distance from the inside wall of the container during normal rotational operation. The end of the shaft may be repeatedly and rapidly “tapped” against the inside wall of the container 6, thereby mimicking the manual tapping often utilized in harvesting cells from the surface of a conventional tissue culture flask and thereby, for example, facilitating cell harvesting from the disks.

Panel A of FIG. 9 shows an example of a cell-culture substrate utilizing embodiments disclosed herein (McMaster-Carr® Part Number 2930T62; 316 Stainless Steel Wire Cloth Disc, 4″ Diameter, 100×100 mesh size). Stainless steel wire “cloth” (as identified in the McMaster-Carr® description) is equivalent to stainless steel wire “mesh,” as the terms “cloth” and “mesh” are generally utilized interchangeably in this case, for example, wherein the mesh is relatively thin and flexible and therefore has handling characteristics of a cloth. In some embodiments, the stainless steel wire mesh substrate may be stiffer (e.g., substantially stiffer) than those utilized in this particular example and therefore more appropriately referred to as mesh instead of cloth. In certain embodiments, a cell-culture substrate may comprise a stainless steel wire cloth disk 1 including openings 2 and wires 3.

Panel B of FIG. 9 shows an example configuration of interleaved growth substrates or disks 4 and spacer substrates or disks 5 on a shaft 6, wherein the growth disks 4 may be selected to have smaller opening sizes and wire diameters than those of the spacer disks 5. Accordingly, the growth disks 4 may have a higher wire surface area per unit planar area relative to the spacer disks 5. Furthermore, a gap or gap distance may be present between the growth disks 4 and the spacer disks 5. The gap may enable, as depicted in FIG. 9C, gas exchange 7 (e.g., periodic gas exchange) and nutrient exchange 8 (e.g., periodic nutrient exchange) with each rotation 9 of the one or more interleaved growth disks 4 and spacer disks 5 through the culture media 10 and the gas headspace 11. The culture media 10 may partially fill the inside of a container (not shown) and the gas headspace 11 may be disposed between the surface of the culture media 10 and an inside surface of a lid (not shown). In some embodiments, during rotary motion 9 of the plurality of one or more cell-culture substrates, the first portion 12 of the surface area of the cell-culture substrates may be outside of the fluid media 10 and in the gas headspace 11. The second portion 13 of the surface area of the one or more cell-culture substrates may remain submerged in the fluid media 10. The surface area of the first portion 12 can vary during the course of the scalable bioreactor process, wherein at some points in the process (e.g., during cell attachment) the surface area of the first portion 12 can be zero, for example, wherein the fluid media covers the entirety of the plurality of one or more cell-culture substrates during the cell attachment process.

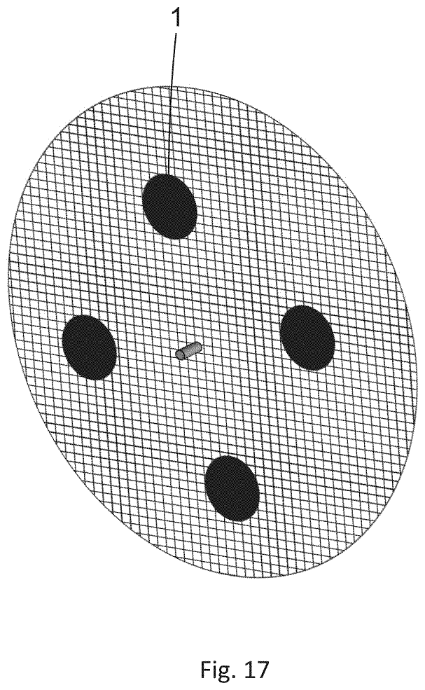

FIG. 17 shows an example of a cell-culture substrate wherein additional larger openings 1 are incorporated to provide additional pathways for nutrient and/or gas exchange. In some cases, one or more cell-culture substrates may incorporate larger holes 1 through the thickness of the cell-culture substrate at prescribed intervals and/or in prescribed patterns. These holes 1 may communicate with corresponding holes in adjacent cell-culture substrates, such as to provide additional pathways for culture medium and/or gas exchange. In some embodiments, the larger holes 1 of a cell-culture substrate may be shifted relative to those of a preceding cell-culture substrate in a stack of cell-culture substrates, thereby providing a curved pathway which, in conjunction with rotation of the stack of cell-culture substrates, may further promote culture medium flow through the stack.

In certain cases, a flow directing or restricting element (e.g., a foil, a baffle, a valve, an orifice, etc.) may be utilized at the entrance of or anywhere within a pathway defined by the larger openings 1 in the cell-culture substrates to promote a desired fluid flow behavior (e.g., unidirectional, laminar, etc.). In various embodiments, as the scale of the planar dimensions of a cell-culture substrate increases (e.g., radius), the distance from the outer edge of the cell-culture substrate to the center of the cell-culture substrate increases and the additional pathways made by larger holes 1 in the cell-culture substrates may be desirable to provide sufficient nutrient and gas exchange. Further to the scalability considerations, a characteristic distance from the bulk culture media within the container to an edge of a planar section of cell-culture substrate may exist within which the balance of mass transport and cell growth surface area may be optimal.

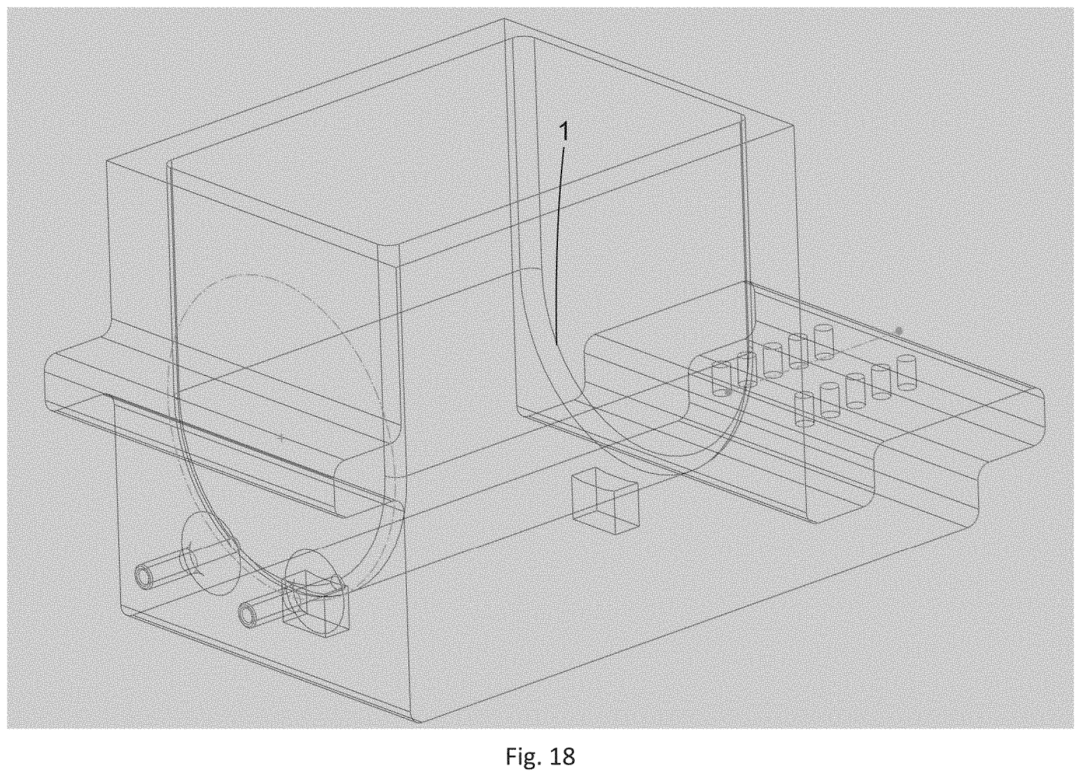

FIG. 18, shows an example of a bioreactor container in which the inside surface 1 of the container substantially conforms to the shape and size of the plurality of cell-culture substrates. Such a configuration may minimize the hold-up volume of the container, increase the efficiency of cell seeding, etc.

In some embodiments, the seeding and proliferation protocols described herein can be performed using a serum-free culture media instead of 10% FBS DMEM/F12. The stack of cell culture substrates can be coated prior to seeding with, for example, fibronectin, gelatin, or any one of a number of cell adhesive coatings to enhance cell attachment.

In certain embodiments, any of the scalable bioreactors described herein may be configured with a bubble diffuser below the stack of cell-culture substrates. Air containing CO₂ (e.g., 5% CO₂) may be supplied through the bubble diffuser during any phase of culture, including, for example, during seeding, proliferation, differentiation, and/or harvest, to provide cells with supplementary oxygen and/or CO₂ removal.

In various embodiments, the media in the bioreactor may be agitated via a magnetic stir bar located below the stack of cell-culture substrates to enhance gas transport to and from the cells. In some embodiments, the bioreactor may be configured with one or more of any type of agitator, mixer, or agitation/mixing feature or mechanism, including, but not limited to, impellers, propellers, fins, baffles, etc. The one or more agitators, mixers, or agitation/mixing features or mechanisms may be configured to rotate and/or move by virtue of the motion of the disk stack. The one or more agitators, mixers, or agitation/mixing features or mechanisms may be configured to rotate and/or move independent of the motion of the disk stack (for example, using a separate motor). In some embodiments, the one or more agitators, mixers, or agitation/mixing features or mechanisms may be confirmed to perform the agitation/mixing function without rotating or moving (e.g., for example, one or more baffles)

In some embodiments, high-level O₂ (e.g., greater than 21% O₂) may be supplied to the headspace of the bioreactor to provide supplementary oxygen to the cells. In certain embodiments, gas transport may be further regulated by continuously or intermittently changing the media height, as disclosed herein in reference to FIG. 7.

In certain embodiments, the bioreactor cell seeding protocol may comprise one or more phases. The cell seeding protocol may comprise two phases, the first of which may last from 1 minute to 48 hours, during which the stack of cell-culture substrates does not rotate (i.e., remains static). In some embodiments, the first phase may be 1 minute to 48 hours, 1 minute to 24 hours, 1 minute to 12 hours, 1 minute to 6 hours, 1 minute to 1 hour, 10 minutes to 48 hours, 10 minutes to 24 hours, 10 minutes to 12 hours, 10 minutes to 6 hours, 10 minutes to 1 hour, 30 minutes to 48 hours, 30 minutes to 24 hours, 30 minutes to 12 hours, 30 minutes to 6 hours, 30 minutes to 1 hour, or any other suitable period of time. The second phase may be 1 second to 48 hours, 1 minute to 24 hours, 1 minute to 12 hours, 1 minute to 6 hours, 1 minute to 1 hour, 10 minutes to 48 hours, 10 minutes to 24 hours, 10 minutes to 12 hours, 10 minutes to 6 hours, 10 minutes to 1 hour, 30 minutes to 48 hours, 30 minutes to 24 hours, 30 minutes to 12 hours, 30 minutes to 6 hours, 30 minutes to 1 hour, or any other suitable period of time, the stack of cell-culture substrates may rotate at a speed of 0.1 to 20, 0.1 to 15, 0.1 to 10, 0.1 to 5, 0.1 to 1, Ito 20, Ito 15, Ito 10, Ito 5 RPM, or any other suitable RPM. The rotation may be either in one direction (i.e., clockwise or counterclockwise) or in changing directions (i.e., clockwise-counterclockwise). The rotation may change intermittently or periodically a plurality of times throughout the duration of the phase (for example, in the case of periodically changing directions, once every 1 second to 24 hours).

In various embodiments, one or more solutions of cells may be utilized in association with the cell-seeding protocol. For example, two cell suspensions may be prepared for bioreactor seeding, the first comprising a range of 1 to 50% of the total target number of cells to be seeded and the second comprising 50 to 99% of the total target number of cells to be seeded. The volume of the first cell suspension added to the bioreactor may be configured such that the stack of cell-culture substrates is fully submerged when the suspension is added. Then, the second cell suspension, comprising a substantially smaller volume than that of the first cell suspension may be added to the bioreactor, on top of the first cell suspension. After a total time of, for example, from 1 minute to 48 hours from the time that the first cell suspension was introduced into the bioreactor, the culture medium height may be adjusted down to slightly above the shaft as disclosed herein.

Referring to FIG. 7, in some embodiments, the stack of cell culture substrates may be tilted as disclosed herein during the cell seeding protocol. In certain embodiments, one or more of the cell seeding protocols disclosed herein may be utilized separately and individually or in combination with one another.

In various instances, cells capable of up-taking lipids and/or differentiating into adipocytes or adipocyte-like cells can be seeded in the bioreactor. After performing the seeding and proliferation protocols as described herein, the cells may be induced to accumulate lipids via exogenous uptake and/or endogenous synthesis. After the induction, lipid-laden cells may be disassociated from the cell-culture substrate utilizing a dissociation agent (e.g., trypsin and/or collagenase) as described herein. The density of mature adipose tissue can be lower than that of water (i.e., 0.92 to 0.970 g/mL). Thus, lipid-laden cells may float to the top of the bioreactor once dissociated. The dissociated cell fraction can subsequently be collected from the top of the bioreactor, for example, by intermittent and/or continuous harvesting operations, including, but not limited to, decanting, skimming, siphoning, pumping, overflowing, draining, etc.

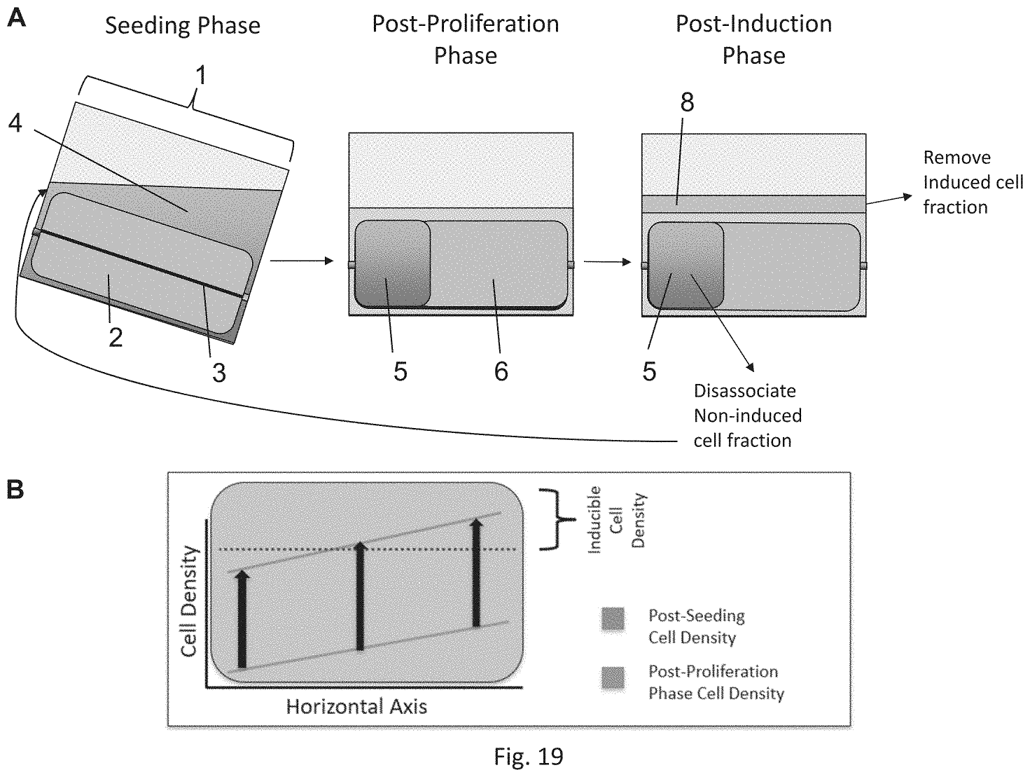

In some embodiments, a semi-continuous process may be performed as shown in panel A of FIG. 19. During the seeding phase, the bioreactor 1 and stack of cell-culture substrates 2 may be tilted as disclosed herein. The cell suspension solution 4 can thus be unevenly distributed throughout the seeding phase. In the example shown in panel A of FIG. 19, a greater portion of the cell suspension accumulates on the right-hand side of the bioreactor. Cells above the stack of cell culture substrate stack fall and attach onto the substrate stack during the seeding process. The tilt can result in an uneven cell distribution across the horizontal axis 3 of the substrate stack as shown in panel B of FIG. 19. Cell density can increase as one moves across the horizontal axis to the right. A cell proliferation phase can be performed as described herein. The resultant cell density post-proliferation can vary across the horizontal axis of the substrate stack, due, for example, to initial differences in the seeding density across the horizontal axis. Cells can be induced to uptake lipids; however, only adequately dense cell regions may be susceptible to lipid accumulation, as cell cycle arrest can mark the first step of differentiation, which itself can occur via cell-to-cell contact inhibition. The region of cells dense enough to undergo lipid uptake is shown as region 6 in panel A of FIG. 19 and is represented schematically in the region above the dotted line in the cell density/horizontal axis plot shown in panel B of FIG. 19. The region of cells not dense enough to undergo differentiation is shown in region 5 in panel A of FIG. 19 and is represented in the region below the dotted line in the cell density/horizontal axis plot shown in FIG. 19, panel B. In this example, the induced cell fraction can result in self-dissociation. The floating dissociated cell fraction 8 can be removed as described herein. The remaining non-induced cell fraction can then be dissociated with trypsin or collagenase as described herein. The dissociated cells can then be used to re-seed the bioreactor and the process can be repeated (e.g., 1, 2, 3, 4, 5, 6, 7, 8, 9, 10, or more times).

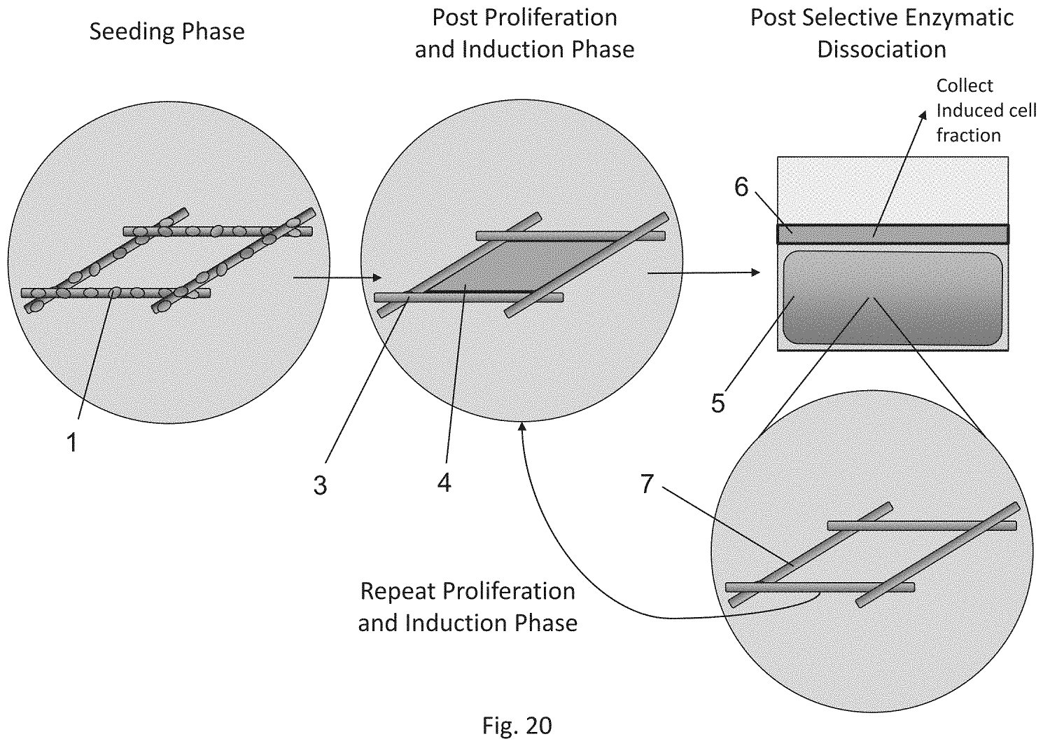

In some embodiments, a semi-continuous process can performed as shown in FIG. 20, which shows both a simplified schematic of the bioreactor system described herein (right-hand box) as well as close-up representations of the cell-culture substrate (e.g., stainless-steel wire mesh) located within the bioreactor (shown in circles). In this example, the wires are coated with fibronectin, which has been previously shown to inhibit adipogenesis. Alternatively, the wire curvature, stiffness and/or curvature of the wires may be chosen so as to prevent differentiation without the use of a coating. Cells capable of accumulating lipids 1 can subsequently be seeded onto a stack of cell-culture substrates using the seeding phase protocols described herein. During the proliferation phase, cells can proliferate both on the wires (i.e., the confluent wires 3) as well as into the openings between the wires (i.e., 4). Cells can subsequently be induced to undergo lipid accumulation. In this example, the cells directly contacting the wire do not accumulate lipid, due to one or more of the inhibitory mechanisms described herein. However, the cells growing into the openings can undergo lipid accumulation due to minimal contact with the wires.

Due to the fibronectin coating on the wire mesh, cells directly attached to the wire mesh may not be as susceptible to collagenase dissociation as cells growing within the wire openings, thus a partial collagenase dissociation can be performed to selectively remove this cell population. The dissociated cell fraction can float to the top of the bioreactor as described herein. The floating dissociated cell fraction 6 can be removed as described herein. Post-selective dissociation, there can still be non-induced cells adhered to the mesh (represented by cylinder 5 and the wire mesh 7 close-up). Subsequent proliferation phases can be performed in which the cells proliferate into the wire openings, can be induced, and after which the harvest procedure can be repeated.

### EXAMPLES

The following illustrative examples are representative of embodiments of the devices and methods described herein and are not meant to be limiting in any way.

### Example 1—Scalable Bioreactor System

A scalable bioreactor system as provided herein may be utilized to expand a number of fibroblast-like animal cells (e.g., derived from duck, swine, bovine, etc.). Moreover, the scalable bioreactor system may be utilized to differentiate the cells for the purpose, for example, of producing a cell-based fat product. The scale of the bioreactor system may be configured or designed for the use of producing fat. The fat may be for use in product development, including tastings, as well as in part of a seed train for cell expansion. A bioreactor scale may be expressed in terms of total cell-culture substrate growth area, and may be, for example, at least 20,000, 30,000, 40,000 cm² or other suitable area.

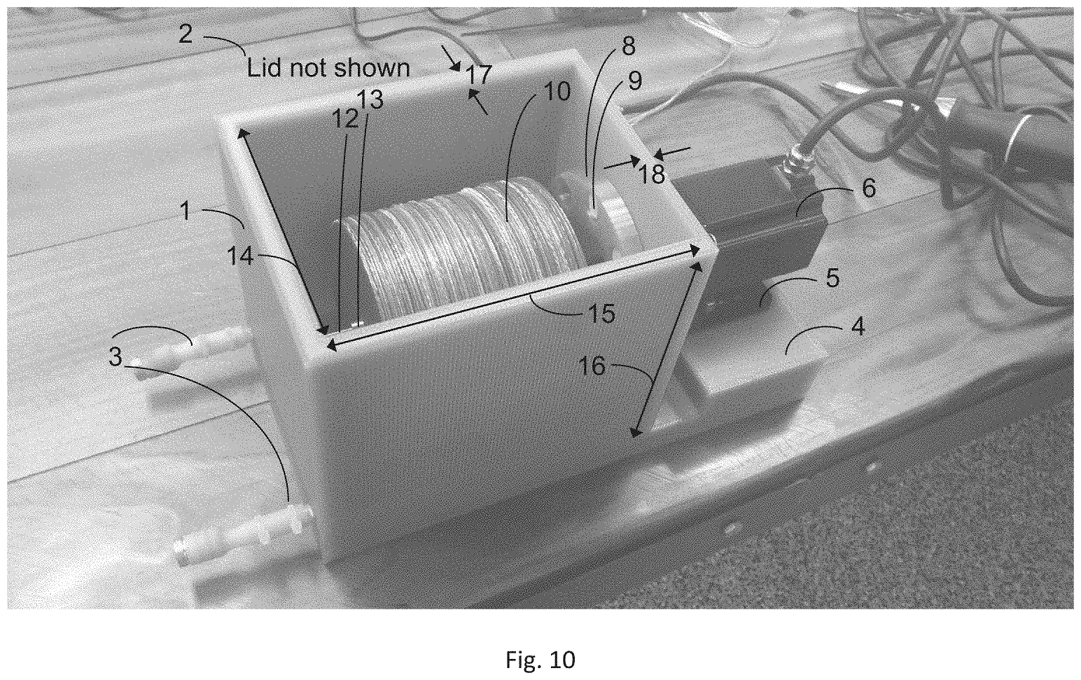

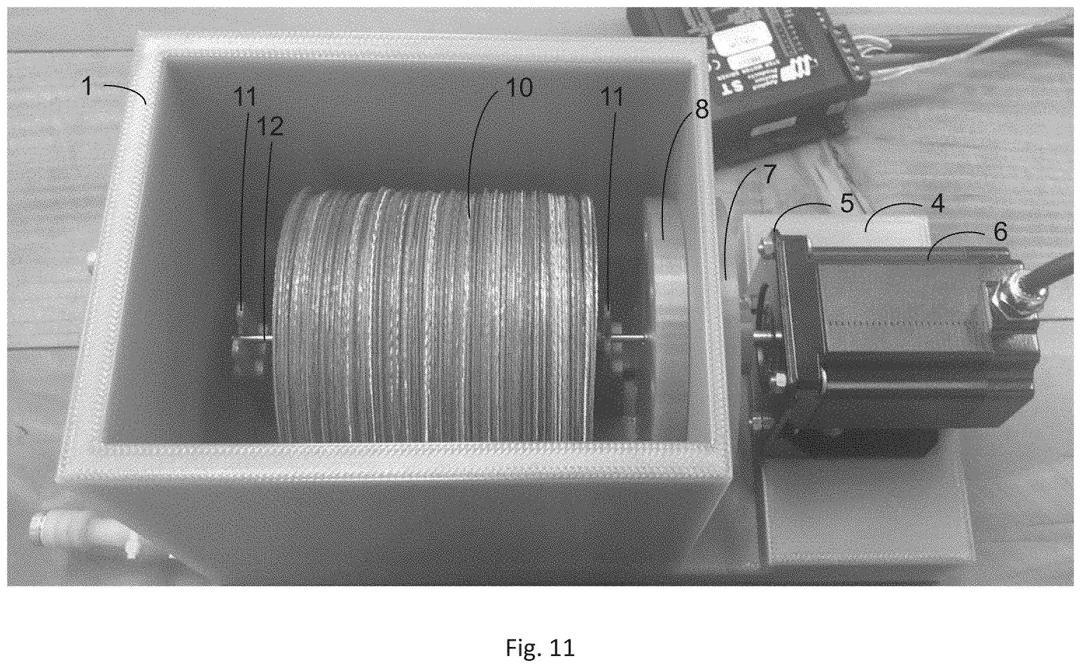

FIG. 10 is a photograph of an example of a scalable bioreactor system as disclosed herein, wherein the same numbering convention utilized in FIG. 1 is employed. In addition to select features called out and defined is FIG. 1, an inside width 14, an inside length 15, an inside height 16, a side wall thickness 17, and a wall thickness 18 between the external magnetic wheel and internal magnetic wheel are indicated. FIG. 11 is a photograph from a substantially top-view of an example of a scalable bioreactor system as disclosed herein, wherein the numbering convention used in FIG. 1 is also employed.

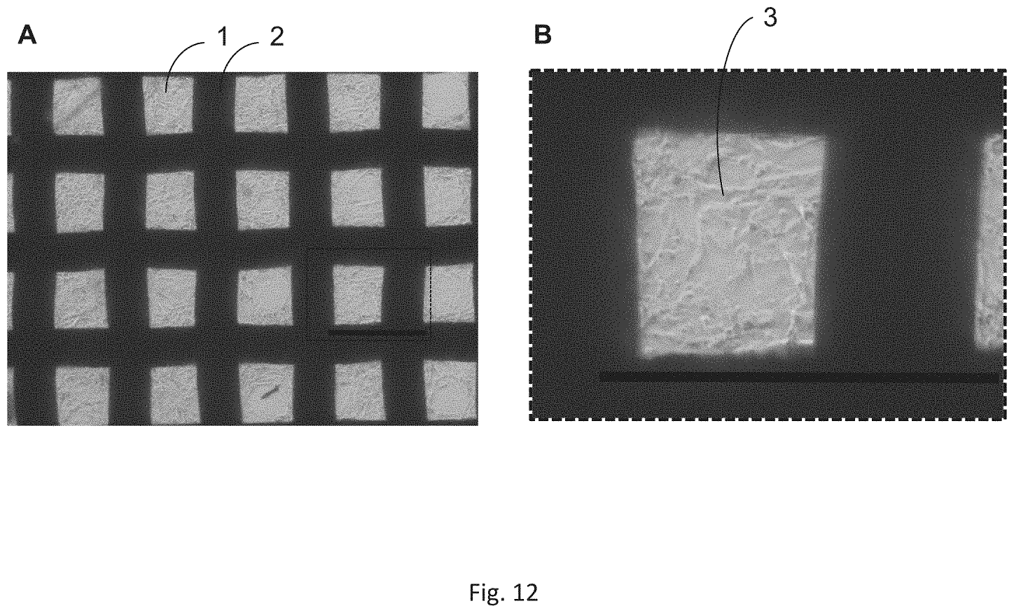

Referring to FIGS. 10 and 11, an example of a scalable bioreactor system as disclosed herein is shown. The scalable bioreactor system includes a container 1, at least two openings 3 (i.e., tubing fittings for culture-media exchange), a feature 4 (i.e., for supporting the stepper motor bracket and stepper motor), an external magnetic wheel (such as the external magnetic wheel 7 depicted in FIG. 1), an internal magnetic wheel 8, and shaft supports (such as shaft supports 11 depicted in FIG. 1). The components of the scalable bioreactor system depicted in FIGS. 11 and 12 were designed utilizing SolidWorks computer-aided design (CAD) software and 3-D printed from the thermally stable (e.g., autoclave sterilizable), biocompatible, FDA-compliant, food contact-approved polymer Ultem™ 1010 polyetherimide by way of a fused deposition modeling (FDM) process (Purple Porcupine; Irvine, Calif.).

Due to the line-by-line extrusion of polymer filaments intrinsic to FDM 3-D printing, such 3-D printed parts may not be inherently water-tight. Accordingly, a solution was developed to seal the bioreactor, rendering it substantially water-tight, while retaining the desired thermal, biocompatibility, FDA-compliance, and food-contact approval characteristics of the Ultem™ 1010 polyetherimide material of construction. The solution included applying a conformal coating of thermally stable, biocompatible, FDA-compliant, food contact-approved polymer parylene (Parylene Engineering, Inc.; San Clemente, Calif.). In this example, the thickness of the parylene coating was chosen to be 40 to 70 microns.

With continued reference to FIGS. 10 and 11, the inside dimensions of the container were 12 cm in width (14) by 16 cm in length (15) by 14 cm in height (16). The wall thicknesses of the container were 1 cm on the side walls (17) and wall farthest from the internal and external magnetic wheels, 0.5 cm on the wall through which the magnetic wheels transmit torque (18), which was designed to be relatively thin. Such a configuration enables the internal and external magnet wheels and associated magnets to be positioned as close as possible to each other without sacrificing the structural integrity of the container so as to facilitate magnetic force and torque transmission from the external magnetic wheel to the internal magnetic wheel. The wall thickness was 2 cm on the bottom, except for 1 cm along a centrally designed 2 cm wide by 16 cm long by 1 cm deep channel for use in adjusting the position of the shaft supports (not visible).

In this example, 0.5″ diameter polytetrafluorethylene (PTFE)-coated neodymium 50 disk magnets 9 (TEF D0050; SuperMagnetMan, Inc., Pelham, Ala.) were press fit into the eight holes in the external magnetic wheel and eight corresponding holes in the internal magnetic wheel 8 (three magnets were press fit into each hole), with their north and south poles facing one another, to enable attractive magnetic force between the magnets in the internal and external magnetic wheels and thereby torque transmission between the external and internal magnetic wheels when the stepper motor 6 turns. In this example, the shaft 12 comprised a 6″ long by ⅛″ diameter 316 stainless steel shaft (Part Number 1263K38; McMaster-Carr®, Elmhurst, Ill.) and the shaft collars 13 comprised ¼″ wide by ⅜″ outside diameter 316 stainless steel shaft collars designed for ⅛″ diameter shafts (Part Number 9943K13; McMaster-Carr®, Elmhurst, Ill.).

The stack of cell-culture substrates 10 comprised a stack of two hundred (200) interleaved 316 stainless steel wire cloth disks, in which every other cell-culture substrate in the stack (i.e., one hundred (100) of the total of two hundred (200) cell-culture substrates) comprised a 4″-diameter, 100×100 mesh size, 316 stainless steel wire cloth disk with an opening size of 0.0055″ (i.e., 140 microns×140 microns) and wire diameter of 0.0045″ (i.e., 114 microns; Part Number 2930T62; McMaster-Carr®, Elmhurst, Ill.). The 100×100 mesh size disks are hereby referred to as “growth disks.” The other one hundred (100) of the total of two hundred (200) cell-culture substrates comprised a 4″-diameter, 20×20 mesh size, 316 stainless steel wire cloth disk with an opening size of 0.034″ (i.e., 813 microns×813 microns) and wire diameter of 0.016″ (i.e., 406 microns; Part Number 2930T23; McMaster-Carr®, Elmhurst, Ill.). The 20×20 mesh size disks are hereby referred to as “spacer disks” (20×20 mesh indicates that in one linear inch, there are 20 openings).

A stack of cell-culture substrates may include 5 to 10,000, 5 to 9,000, 5 to 8,000, 5 to 7,000, 5 to 6,000, 5 to 5,000, 5 to 4,000, 5 to 3,000, 5 to 2,000, 5 to 1,000, 5 to 900, 5 to 800, 5 to 700, 5 to 600, 5 to 500, 5 to 400, 5 to 300, 5 to 200, 5 to 100, 5 to 50, 5 to 25, 50 to 10,000, 50 to 9,000, 50 to 8,000, 50 to 7,000, 50 to 6,000, 50 to 5,000, 50 to 4,000, 50 to 3,000, 50 to 2,000, 50 to 1,000, 50 to 900, 50 to 800, 50 to 700, 50 to 600, 50 to 500, 50 to 400, 50 to 300, 50 to 200, 50 to 100, 50 to 75, 50 to 60, 100 to 10,000, 100 to 9,000, 100 to 8,000, 100 to 7,000, 100 to 6,000, 100 to 5,000, 100 to 4,000, 100 to 3,000, 100 to 2,000, 100 to 1,000, 100 to 900, 100 to 800, 100 to 700, 100 to 600, 100 to 500, 100 to 400, 100 to 300, 100 to 200, 1,000 to 100,000, 1,000 to 90,000, 1,000 to 80,000, 1,000 to 70,000, 1,000 to 60,000, 1,000 to 50,000, 1,000 to 4,000, 1,000 to 30,000, 1,000 to 20,000, 1,000 to 10,000, 1,000 to 9,000, 1,000 to 8,000, 1,000 to 7,000, 1,000 to 6,000, 1,000 to 5,000, 1,000 to 4,000, 1,000 to 3,000, 1,000 to 2,000, or any other suitable number of cell-culture substrates. A stack of cell-culture substrates may include 5 to 10,000, 5 to 9,000, 5 to 8,000, 5 to 7,000, 5 to 6,000, 5 to 5,000, 5 to 4,000, 5 to 3,000, 5 to 2,000, 5 to 1,000, 5 to 900, 5 to 800, 5 to 700, 5 to 600, 5 to 500, 5 to 400, 5 to 300, 5 to 200, 5 to 100, 5 to 50, 5 to 25, 50 to 10,000, 50 to 9,000, 50 to 8,000, 50 to 7,000, 50 to 6,000, 50 to 5,000, 50 to 4,000, 50 to 3,000, 50 to 2,000, 50 to 1,000, 50 to 900, 50 to 800, 50 to 700, 50 to 600, 50 to 500, 50 to 400, 50 to 300, 50 to 200, 50 to 100, 50 to 75, 50 to 60, 100 to 10,000, 100 to 9,000, 100 to 8,000, 100 to 7,000, 100 to 6,000, 100 to 5,000, 100 to 4,000, 100 to 3,000, 100 to 2,000, 100 to 1,000, 100 to 900, 100 to 800, 100 to 700, 100 to 600, 100 to 500, 100 to 400, 100 to 300, 100 to 200, 1,000 to 100,000, 1,000 to 90,000, 1,000 to 80,000, 1,000 to 70,000, 1,000 to 60,000, 1,000 to 50,000, 1,000 to 4,000, 1,000 to 30,000, 1,000 to 20,000, 1,000 to 10,000, 1,000 to 9,000, 1,000 to 8,000, 1,000 to 7,000, 1,000 to 6,000, 1,000 to 5,000, 1,000 to 4,000, 1,000 to 3,000, 1,000 to 2,000, or any other suitable number of growth substrates. A stack of cell-culture substrates may include 5 to 10,000, 5 to 9,000, 5 to 8,000, 5 to 7,000, 5 to 6,000, 5 to 5,000, 5 to 4,000, 5 to 3,000, 5 to 2,000, 5 to 1,000, 5 to 900, 5 to 800, 5 to 700, 5 to 600, 5 to 500, 5 to 400, 5 to 300, 5 to 200, 5 to 100, 5 to 50, 5 to 25, 50 to 10,000, 50 to 9,000, 50 to 8,000, 50 to 7,000, 50 to 6,000, 50 to 5,000, 50 to 4,000, 50 to 3,000, 50 to 2,000, 50 to 1,000, 50 to 900, 50 to 800, 50 to 700, 50 to 600, 50 to 500, 50 to 400, 50 to 300, 50 to 200, 50 to 100, 50 to 75, 50 to 60, 100 to 10,000, 100 to 9,000, 100 to 8,000, 100 to 7,000, 100 to 6,000, 100 to 5,000, 100 to 4,000, 100 to 3,000, 100 to 2,000, 100 to 1,000, 100 to 900, 100 to 800, 100 to 700, 100 to 600, 100 to 500, 100 to 400, 100 to 300, 100 to 200, 1,000 to 100,000, 1,000 to 90,000, 1,000 to 80,000, 1,000 to 70,000, 1,000 to 60,000, 1,000 to 50,000, 1,000 to 4,000, 1,000 to 30,000, 1,000 to 20,000, 1,000 to 10,000, 1,000 to 9,000, 1,000 to 8,000, 1,000 to 7,000, 1,000 to 6,000, 1,000 to 5,000, 1,000 to 4,000, 1,000 to 3,000, 1,000 to 2,000, or any other suitable number of spacer substrates.

The opening dimensions and wire dimensions of the growth disks were chosen to be substantially similar to the opening size and structural element dimensions of lattice-like tissue-engineering scaffolds (e.g., 200 micron×200 micron opening size (i.e., pore size) and 100-micron wide structural elements.

A larger mesh size (e.g., 100×100 mesh size) growth disk (e.g., growth substrate) and a smaller mesh size (e.g., 20×20 mesh size) spacer disk (e.g., spacer substrate) were chosen such that the larger openings of the spacer disks provide a path for nutrient exchange (i.e., culture media) and gas exchange (e.g., oxygen, CO₂, etc.) along the radial direction of the disks, thereby periodically allowing culture media to drain from and then bath a substantial portion of the planar area of both the 100×100 mesh size and 20×20 mesh size disks with each rotation of the stack of disks through the culture media partially filling the inside of the container (e.g., substantially covering half of the diameter of the disks) and the 5% CO₂ air headspace above the media, between the surface of the culture media and the inside of the lid.

Further to the utility of the cell-growth substrates utilized in this example, the wire surface area (i.e., the area available for initial cell attachment and proliferation) of the 316 stainless steel wires comprising the 4″-diameter wire cloth disks was estimated by making the simplifying assumption that the structure of the woven wire cloth could be approximated by a lattice of overlapping wires having substantially the same opening size and wire diameter. The estimated wire surface area of the growth disks was estimated by multiplying the estimated number of wires within a 1 cm² unit area (i.e., 40×2) by the estimated surface area of the wires (i.e., assuming cylindrical, with length 1 cm, diameter 114 microns, and estimated surface area per wire of 0.036 cm²/cm length of wire and was found to be 2.87 cm² of wire surface area per unit of disk planar area (i.e., per unit of cell-culture substrate planar area)). Hence, the estimated wire surface area available for initial cell attachment and proliferation was estimated to be 233 cm² per 4″-diameter growth disk (i.e., 10.16 cm diameter and 81.1 cm² planar area). Calculated similarly, the estimated wire surface area of the spacer disks was estimated to be 2.04 cm² of wire surface area per unit of disk planar area, alternatively expressed as 165 cm² of wire surface area per the 4″-diameter spacer disk. Therefore, in this example, the average wire surface area of the stack of interleaved growth disks and spacer disks (i.e., the cell-culture substrates) is estimated as 2.455 cm² of wire surface area per disk planar area and therefore 199 cm² per 4″-diameter disk (i.e., 10.16 cm diameter disk). Therefore, in this example, the stack of 200 interleaved growth and spacer disks (i.e., stack of cell-culture substrates) has an estimated total wire surface area for cell attachment of proliferation of 39,820 cm².

Accordingly, an intention in this example is that the cells attach to and proliferate on the wires of both the growth disks and spacer disks, that the cells grow into and fill in the openings of the growth disks (and to a lesser extent those of the spacer disks, so that pathways for culture media and gas exchange are largely retained), but so that extracellular matrix (ECM) deposition is minimized following the cellular ingrowth into the openings of the growth disks, in such a way as to enable cells to be harvested from the mesh (e.g., by way of an enzyme such as trypsin). In this example, ECM deposition was anticipated to be minimized by controlling the duration of culture, wherein total culture times on the mesh in this example ranged from 4 to 7 days. In the bioreactor described herein, in some embodiments cells may be seeded at a ratio of cells to cell-culture substrate surface area (i.e., the surface area provided by the growth substrates plus the surface area provided by the spacer substrates) ranging from about 500 cells per cm² to about 200,000 cells per cm².

### Example 2—Cell Growth

Panel A of FIG. 12 shows an example phase-contrast light photomicrograph of a 72-hour culture of duck-derived fibroblast-like cells growing into the openings 1. (i.e., pores) of a cell-culture substrate comprising a 1″-diameter 100×100 mesh size 316 stainless steel wire cloth disk. Due to the opacity of the stainless steel wires 2, the cells are not directly visible on the wires 2. The cells were seeded at a density of 13,333 cells/cm on a wire surface area basis. The wire cloth disk had an estimated planar area of 5.07 cm and estimated wire surface area of 14.5 cm. The cell-seeded cell-culture substrate was cultivated at 37° C. and 5% CO in 4 mL of a culture medium comprising 10% FBS in a DMEM/F12 basal medium in a 6-well plate. In this example, it was understood that only about half of the total wire surface area would have been presented facing up toward the cells during their initial gravity-mediated sedimentation down onto the cell-culture substrate. The scale bar is approximately 250 microns. Panel B of FIG. 12 shows a higher magnification of one opening, in which individual cells 3 are more readily distinguishable within the opening.

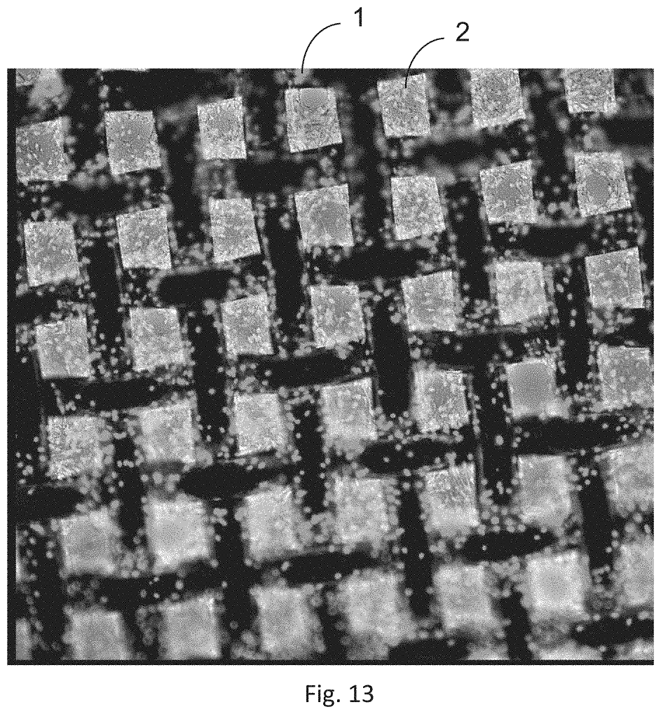

FIG. 13 shows an example fluorescence micrograph of a 96-hour culture of duck-derived fibroblast-like cells growing on the wires and into the openings of a cell-culture substrate comprising a 1″-diameter 100×100 mesh size 316 stainless steel wire cloth disk stained with a Hoechst dye to highlight the cell nuclei. Utilizing Hoechst staining and fluorescence microscopy, cells were identified both on the wires 1 and growing into the openings 2.

Referring to FIGS. 12 and 13, the proliferation of duck-derived fibroblast-like cells was first demonstrated on the 316 stainless steel wire cloth disk cell-culture substrates in small-scale (i.e., 6-well format) proof-of-concept experiments, as observed by phase-contrast light microscopy (FIG. 12) and fluorescence microscopy of Hoechst stained cell nuclei (FIG. 13).

The 316 stainless steel wire cloth disk was chosen as a cell-culture substrate, not only because of its reported utility in high-density cell expansion and similarity to a lattice-like tissue-engineering scaffold reported to support cell growth both on the structural elements of the lattice and within the openings, but also for the 316 stainless steel material of construction's amenability to sterilization, cleaning, reuse, and food-contact approval, which can be useful for industrial applications wherein minimization of consumables and thereby reduction of cost-of-goods can play an important role in the scalability and profitability of the process. Such considerations can be useful in cell expansion for cell therapy, tissue engineering, and cell-based meat and cell-based fat applications. The 316 stainless steel wire mesh disks were cleaned in an ultrasonic bath using 70% isopropyl alcohol, rinsed with deionized water, dried, and steam sterilized in an autoclave.

⅛″ diameter holes were drilled into the substantial center of each of the 200 cell-culture substrates and the 200 cell-culture substrates were disposed onto the shaft by pushing the shaft through the drilled holes. The 200 cell-culture substrates were compressed together utilizing conventional c-clamps, until they were substantially tightly packed, and then the set screws of the shaft collars were tightened, thereby retaining the 200 cell-culture substrates in a stack ranging in length from 10 to 11.5 cm long, as estimated by measuring with a conventional ruler. Notably, in this example, in which 4″-diameter 100×100 mesh size growth disks were interleaved with 4″-diameter 20×20 mesh size spacer disks, the thickness of each disk was estimated to be twice the reported wire diameter (i.e., the thickness of each growth disk was estimated as 228.6 microns and the thickness of each spacer disk was estimated as 812.8 microns). Thus, the estimated length of the stack of the 200 interleaved growth disks and spacer disks was 10.4 cm.

The bioreactor components were autoclave sterilized separately, assembled aseptically within a biological safety cabinet, wrapped in sterilization wrap, and then re-sterilized by autoclave at 121° C. with a 30 minute dwell time.

### Example 3—Seeding of the Bioreactor

The bioreactor cell seeding was accomplished by first generating a suspension of approximately 80 million duck-derived fibroblast-like cells in a culture medium comprising 10% Fetal Bovine Serum (FBS) in Dulbecco's Modification of Eagle's Medium (DMEM)/F12 basal medium to provide nutrients and growth factors for cell maintenance and growth as well as cell-adhesive proteins (e.g., fibronectin present in the FBS) capable of coating the growth substrates and spacer substrates and thereby facilitating cell attachment. The culture medium also included 1% penicillin-streptomycin as an antibiotic. Within a biological safety cabinet, the lid was removed from the autoclave-sterilized bioreactor container and the cell suspension was manually poured onto a stack of 200×4″-diameter 316 stainless steel wire cloth disks, moving the mouth of the bottle of cell suspension back and forth over the length of the stack of disks. After approximately 4 hours, the culture medium height was adjusted down to slightly above the shaft (i.e., about 2 mm above the shaft, covering substantially half of the planar area of the 4″-diameter disks). The culture was maintained for a period of 4 days, during which time the stack of disks rotated through the medium at a rate of about 1 RPM with culture medium exchanges about once per day, dependent on the qualitatively assessed color of the culture medium (i.e., with visual, unaided-eye color changes from red to orange to yellow being evidence of progressive metabolism of the culture medium and associated lower pH values associated with lactic acid accumulation in the culture medium).

At the end of culture, duck-derived fibroblast-like cells were harvested from the 316 stainless steel wire cloth disks by utilizing a 0.25% trypsin-EDTA solution at room temperature to 37° C. For some stainless steel wire cloth disks, the disks were submerged in 0.25% trypsin-EDTA solution overnight (i.e., approximately 16 hours) at 2-8° C. in a refrigerator prior to bringing the solution up to room temperature to 37° C. The overnight 2-8° C. treatment of the 316 stainless steel wire cloth disks and associated adherent animal cells was found to facilitate cell harvesting. Cells were subsequently centrifuged, and pellets collected.

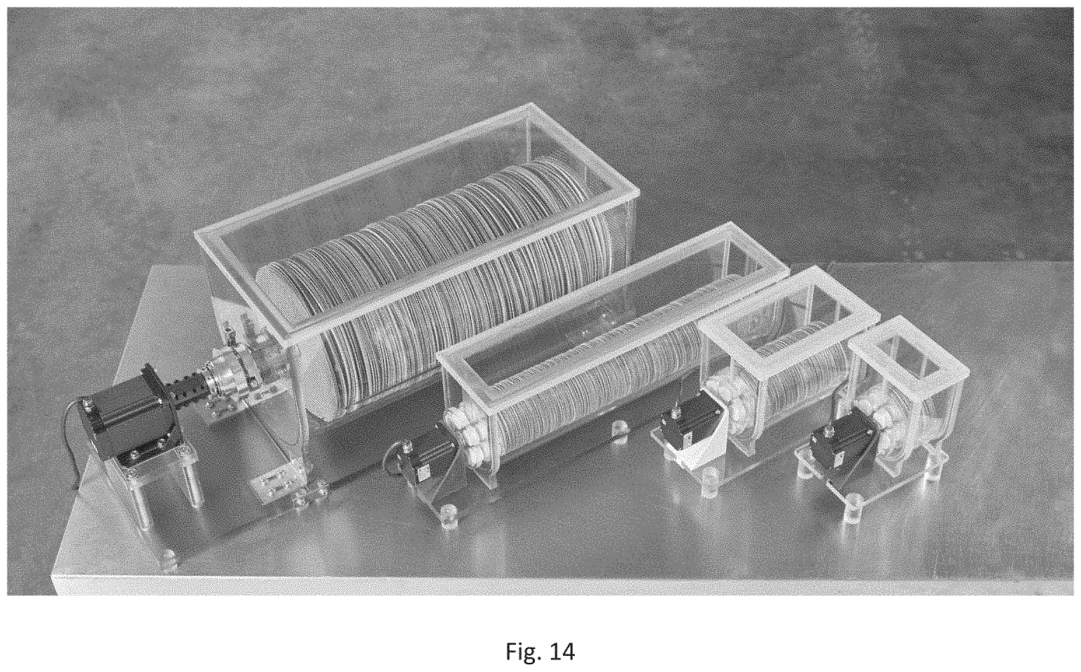

Referring to FIG. 14, in some embodiments, the scalable bioreactor systems described herein were fabricated, as disclosed herein, from polycarbonate. In this photograph, four different scales of the scalable bioreactor systems disclosed herein are shown (e.g., larger to smaller bioreactors).

### Example 4—Measuring of Porcine-Adipose Cell Proliferation

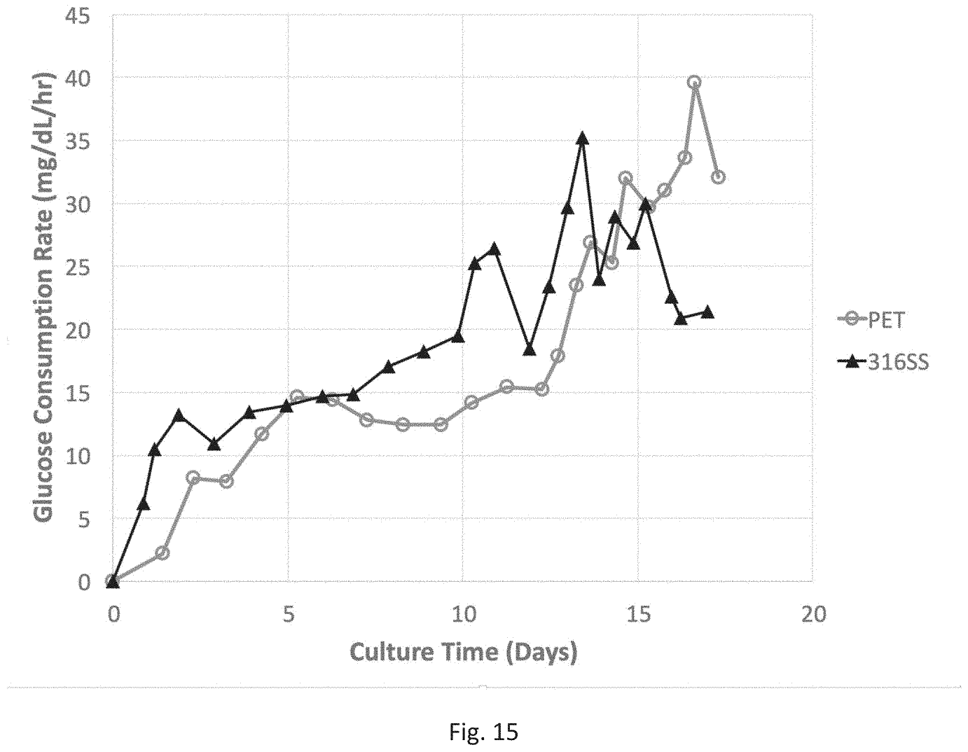

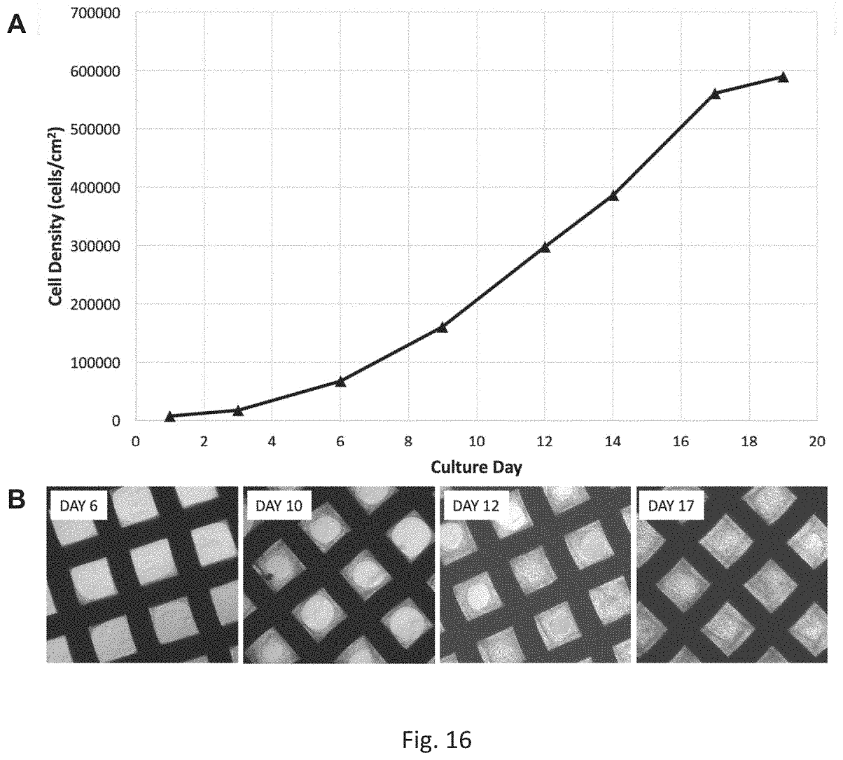

Referring to FIG. 15, in some embodiments, cell proliferation was monitored indirectly by monitoring glucose consumption rates throughout the process. The glucose consumption rates (mg/dL/hr) are plotted as a function of culture time (days) for bioreactor batches in which the cell-culture substrates comprised polyester (i.e., polyethylene terephthalate (PET)) and 316 stainless steel mesh disks. In this example, for the PET polyester mesh-based bioreactor, the growth substrates comprised a total of thirty seven 4″-diameter 46×46 mesh size disks and the spacer substrates comprised a total of thirty seven 2″-diameter 20×20 mesh size disks. In this example, for the 316 stainless steel mesh-based bioreactor, the growth substrates comprised a total of thirty two 4″-diameter 40×40 mesh size disks and the spacer substrates comprised a total of thirty two 3″-diameter 20×20 mesh size disks. In this example, the cell-culture substrates were seeded with porcine adipose-derived cells at a target density of 30,000 cells/cm² of wire surface area. Referring to FIG. 16, cell proliferation was monitored via removal of one or more disks from the stack of cell-culture substrates and/or via smaller scale (e.g., 0.5″ diameter) mesh disks cultivated in parallel with the bioreactor batch in 24-well plates. Referring to panel A of FIG. 16, the cell density (cells/cm²) was measured as a function of culture day by harvesting cells from the cell-culture substrates as described herein. Removed disks were subsequently imaged by phase-contrast light microscopy (e.g., FIG. 16, panel B) and/or stained with Hoechst and observed and/or imaged via fluorescence microscopy.

### Example 5—Analyzing Surface Areas of Cell-Growth Substrates

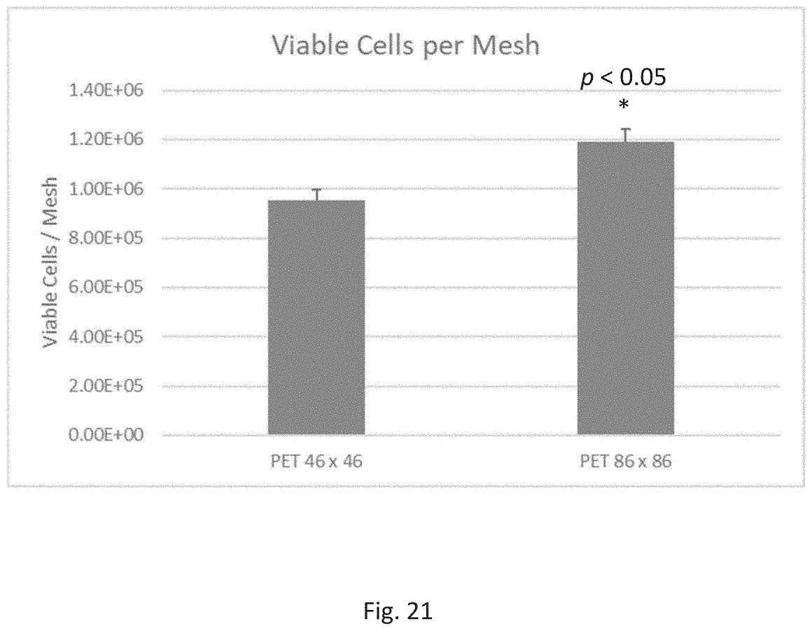

Referring to FIG. 21, an example of a useful finding is that configurations of cell-growth substrates comprising a smaller surface area are capable of yielding higher densities of porcine (i.e., pig) adipose-derived cells per unit area. For example, in some embodiments, meshes comprising different numbers of wires or openings per inch (N×N, where N represents a number of openings per inch) and associated surface areas of wire (e.g., 46×46 and 86×86) yielded different cell numbers, with the cell densities being inversely proportional to the surface area of wire cell-growth substrate (i.e., 86×86 had the smallest surface area of wire, about 1.62 cm² of cell-growth area per cm² of planar area, and 46×46 had the highest surface area of wire, about 2.14 cm² of cell-growth area per cm² of planar area). In this example, the growth substrates were 0.5 inches in diameter and the spacer substrates were 0.5 inches in diameter. This may be useful from a scalability and manufacturing perspective in that the quantities and associated costs of coating the meshes is less due to the lower surface area to be coated. These findings demonstrate the advantages and benefits of the bioreactor systems disclosed herein, which can yield greater cell growth in more compact volumes, and using fewer resources or materials.

FIG. 21 indicates that a more porous mesh having a lower available surface area for cell growth can yield a higher cell growth density compared to a less porous mesh having a larger available surface area for cell growth. The cell growth density may be inversely proportional to a porosity of the mesh, which suggests that cell growth density may be a function of mesh porosity (i.e., a number of openings per unit area, or a ratio of an open area of the mesh to a total area of the mesh) and/or the total available surface area for cell growth or cultivation. A more porous mesh having a lower available surface area may require less materials to manufacture, which means that more cells can be grown or cultivated using fewer resources or materials.

In an aspect, the present disclosure provides a cell cultivation system comprising at least one growth substrate for growing one or more cell types. The at least one growth substrate may have a mesh porosity and a surface area such that a higher cell growth density per unit area is achieved when using a growth substrate that has a higher mesh porosity and a smaller surface area than another growth substrate with a lower mesh porosity and a greater surface area.

In some embodiments, the mesh porosity and the surface area per unit area may be based on a number of openings per unit length or area in the at least one growth substrate. In some cases, the openings may be arranged in a repeating pattern across a surface of the at least one growth substrate. Alternatively, the openings may be arranged in a non-repeating (i.e., random) or predetermined spatial configuration to provide different porosities and surface areas per unit area in different regions or portions of the growth substrate.

In some embodiments, the cell growth density per unit area on the at least one growth substrate may increase as the number of openings per unit dimension or area increases. In some instances, the cell growth density per unit area on the at least one growth substrate may increase by a first amount (e.g., at least 5%) when the number of openings is increased by a second amount (e.g., at least 10%). The first amount and the second amount may be equal. The first amount and the second amount may be different. In some cases, the first amount and the second amount may be linearly proportional. In other cases, the first amount and the second amount may not or need not be linearly proportional.

In some embodiments, the cell growth density per unit area on the at least one growth substrate may increase as the surface area for cell growth per unit area decreases. In some instances, the cell growth density per unit area on the at least one growth substrate may increase by a first amount (e.g., at least 5%) when the surface area for cell growth per unit area is decreased by a second amount (e.g., at least 10%). The first amount and the second amount may be equal. The first amount and the second amount may be different. In some cases, the first amount and the second amount may be linearly proportional. In other cases, the first amount and the second amount may not or need not be linearly proportional.

In some embodiments, the system may further comprise at least one spacer substrate adjacent to the at least one growth substrate. The at least one spacer substrate may be used to facilitate transport and distribution of a growth medium over or across a surface of the at least one growth substrate.

### Example 6—Analyzing Diameters of Spacer Substrates

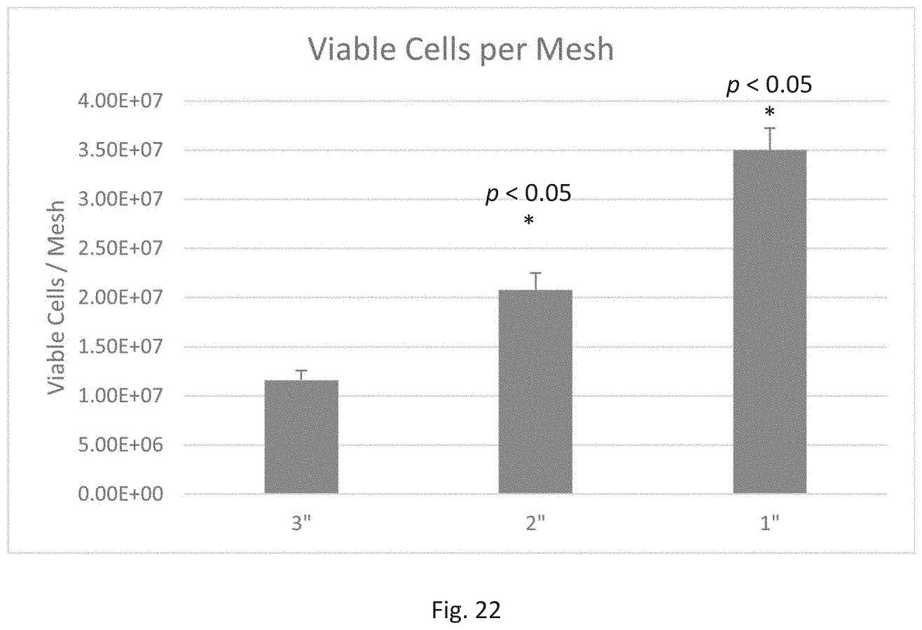

Referring to FIG. 22, an example of a useful finding is that a configuration of substrates comprising smaller diameter spacer substrates is capable of yielding higher cell densities per unit area on the adjacent growth substrates. For example, 20×20 mesh size spacer substrates with diameters of 1″, 2″, and 3″ were configured interleaved with 4″-diameter 40×40 mesh size growth substrates. The 1″ diameter spacer substrates yielded significantly higher numbers of porcine (i.e., pig) adipose-derived cells on the adjacent 4″-diameter growth substrates compared with both the 2″-diameter spacer substrates and the 3″-diameter spacer substrates. This may be useful from a scalability and manufacturing perspective in that smaller diameter spacer substrates result in less material usage and associated weight of the system. These findings demonstrate the advantages and benefits of the bioreactor systems disclosed herein, which can yield greater cell growth in more compact volumes.

FIG. 22 indicates that cell growth density may increase as the size of the spacer substrate (e.g., a dimension such as a length, width, height, thickness, radius, diameter, circumference, etc.) is reduced. In other words, the cell growth density may be inversely proportional to a dimension or an area of the spacer substrate, which suggests that if the spacer substrate is too large, it may impede the flow of the growth medium between the spacer substrate and the growth substrate, thereby reducing cell growth density. A smaller spacer substrate may require less materials to manufacture, which means that more cells can be grown or cultivated in more compact volumes or environments.

In an aspect, the present disclosure provides a cell cultivation system comprising at least one growth substrate for growing one or more cell types and at least one spacer substrate adjacent to the at least one growth substrate. The at least one spacer substrate may be sized to influence a cell growth density on the at least one growth substrate, such that a higher cell growth density per unit area is achieved on the at least one growth substrate when using a smaller spacer substrate compared to a larger spacer substrate.

In some embodiments, a size of the at least one spacer substrate may be based on a dimension as measured along a radial or longitudinal direction of the at least one spacer substrate. In some cases, the cell growth density per unit area on the at least one growth substrate may increase as the dimension of the at least one spacer substrate is reduced along the radial or longitudinal direction. In some instances, the cell growth density per unit area on the at least one growth substrate may increase by a first amount (e.g., at least 5%) when the size of the at least one spacer substrate is decreased by a second amount (e.g., at least 10%). The first amount and the second amount may be equal. The first amount and the second amount may be different. In some cases, the first amount and the second amount may be linearly proportional. In other cases, the first amount and the second amount may not or need not be linearly proportional.

In some cases, the smaller spacer substrate may have a smaller surface area than the larger spacer substrate. In some cases, the smaller spacer substrate may have a same thickness as the larger spacer substrate. In some cases, a size of the at least one growth substrate may be substantially equal to or greater than a size of the at least one spacer substrate. In some embodiments, the cell growth density per unit area may be based at least in part on a ratio between the surface area of a growth substrate and the surface area of a spacer substrate. In some cases, the at least one growth substrate and the at least one spacer substrate may comprise a same number of openings per unit area. In other cases, the at least one growth substrate and the at least one spacer substrate may comprise a different number of openings per unit area.

### Example 7—Rotational Speeds and Cell Yield

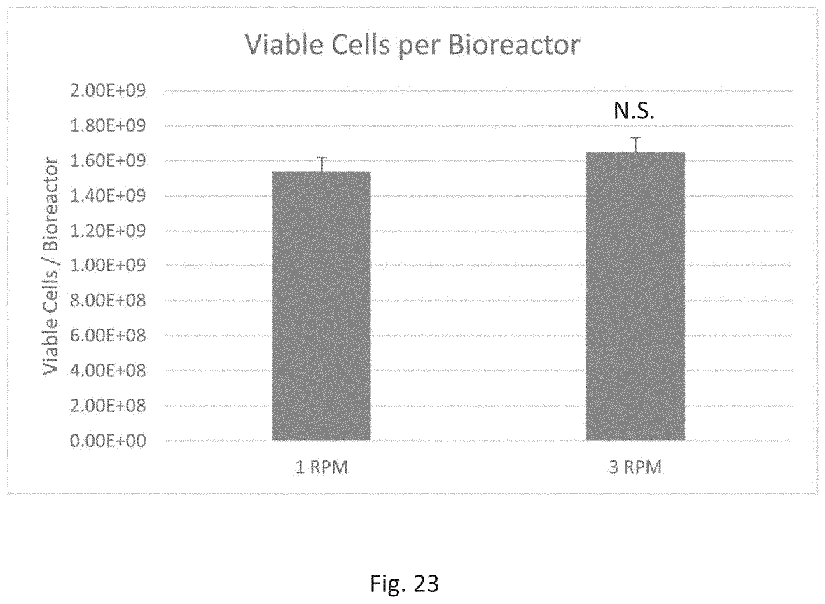

Referring to FIG. 23, an example of a useful finding is that during operation of a bioreactor at rotational speeds ranging from about 1 RPM to about 3 RPM, the yield of porcine (i.e., pig) adipose-derived cells of the bioreactor is not significantly sensitive to or affected by the rotational speed of the bioreactor. This may useful from a scalability and manufacturing perspective in that it offers a broader range of operating parameters within which cell yields can be predictable. These findings demonstrate the advantages and benefits of the bioreactor systems disclosed herein, which can permit consistent or predictable cell growth within a desired accuracy or tolerance under various or varying bioreactor operating conditions.

### Additional Embodiments

In any of the embodiments described herein, the distances between the growth substrates and the spacer substrates may be adjusted manually or automatically based on a quantity or type of material or cells to be grown or cultivated. The distances between the growth substrates and the spacer substrates may be adjusted manually or automatically based on one or more sensor readings associated with a performance or an operation of the bioreactor. The one or more sensor readings may be obtained using a pH sensor, a dissolved oxygen sensor, a temperature sensor, a humidity sensor, a pressure sensor, or any other type of biological, chemical, physical, or optical sensor. The performance or operation of the bioreactor may be associated with an amount of cell growth per unit time or per unit area or volume, or an internal environmental condition or state of the bioreactor.

In any of the embodiments described herein, the positions and/or orientations of the spacers relative to one another may be adjusted manually or automatically (e.g., with aid of one or more motors or actuators) based on a quantity or type of material or cells to be grown or cultivated. The positions and/or orientations of the spacers relative to one another may be adjusted manually or automatically based on one or more sensor readings associated with a performance or an operation of the bioreactor. The one or more sensor readings may be obtained using a pH sensor, a dissolved oxygen sensor, a temperature sensor, a humidity sensor, a pressure sensor, or any other type of biological, chemical, physical, or optical sensor. The performance or operation of the bioreactor may be associated with an amount of cell growth per unit time or per unit area or volume, or an internal environmental condition or state of the bioreactor.

In any of the embodiments described herein, the motor may be configured to rotate at an optimal rate of rotation. The optimal rate of rotation may be adjustable by a user or an operator of the bioreactor. The optimal rate of rotation may be adjusted manually or automatically based on a feedback loop that utilizes one or more sensor readings associated with a performance or an operation of the bioreactor. The feedback loop may comprise a proportional controller, an integral controller, a derivative controller, a proportional integral controller, a proportional derivative controller, an integral derivative controller, or a proportional integral derivative controller. The one or more sensor readings may be obtained using a pH sensor, a dissolved oxygen sensor, a temperature sensor, a humidity sensor, a pressure sensor, or any other type of biological, chemical, physical, or optical sensor. The performance or operation of the bioreactor may be associated with an amount of cell growth per unit time or per unit area or volume, or an internal environmental condition or state of the bioreactor.

In any of the embodiments described herein, the positions and/or orientations of the growth substrates relative to each other may be adjusted manually or automatically based on a quantity or type of material or cell to be grown or cultivated, or based on one or more sensor readings associated with a performance or an operation of the bioreactor. The biological material may comprise cells, tissues, organs, cell-based meat, cell-based fat, or a combination thereof. The rotatable shaft may be coupled to the first growth substrate and the second substrate. The rotatable shaft may be coupled to a first lateral center of the first growth substrate and to a second lateral center of the second growth substrate. The bioreactor may further comprise a motor coupled to the rotatable shaft and configured to rotate the rotatable shaft, thereby rotating the first growth substrate and the second growth substrate. The motor may be selected from the group consisting of a servomotor and a stepper motor. The rotation of the rotatable shaft may be adjusted manually or automatically based on a quantity or type of material or cell to be grown or cultivated, or based on one or more sensor readings associated with a performance or an operation of the bioreactor.

In any of the embodiments described herein, one or more motors (e.g., a servo motor or a stepper motor) may be affixed or coupled to features of the container via brackets or other means and utilized to apply any combination of rotary, angular, or linear motion to the cell-culture substrates inside the container. In some embodiments, a continuous rotary motion at a speed ranging from 0.01 to 1.5 RPM may be applied for a portion of or the entirety of the cell-culture substrate coating or seeding attachment process, the cell cultivation process (i.e., cell proliferation or differentiation), or the cell harvesting process. In some embodiments, an alternating rotary motion may be applied at a speed ranging from 0.01 to 1.5 RPM for a portion of or the entirety of cell-culture substrate coating or seeding attachment process, the cell cultivation process (i.e., cell proliferation or differentiation), or the cell harvesting process. The alternating rotary motion may comprise a rotation in a first direction during a first time period and a rotation in a second direction during a second time period. In some embodiments, the at least one of a plurality of cell-culture substrates are periodically rotated through an air-liquid (e.g., air-culture medium) interface to provide enhanced fluid transport (e.g., liquid and/or gas transport) to and from the adherent cells during each pass through the gas phase, while incurring minimal fluid shear stresses during each pass through and nutrient exchange with the liquid phase. The period of rotation may be set by an operator or automatically adjusted based on a quantity or type of material or cell to be grown or cultivated, or based on one or more sensor readings associated with a performance or an operation of the bioreactor.

In any of the embodiments described herein, a plurality of motors or actuators may be used to adjust a position and/or an orientation of one or more components of the system based on one or more sensor readings. The sensor readings may be obtained using any of the sensors described herein, and may be associated with a performance or an operation of the bioreactors described herein.

Methods of culturing cells are also provided herein. The methods may include culturing cells. The cells may be cultured on cell-culture substrates or growth substrates as provided herein. The cells may be cultured on disk stacks as provided herein. The cell-culture substrates, growth substrates, and/or disk stacks may be disposed in or be a component of a bioreactor. The cell-culture substrates, growth substrates, and/or disk stacks may be disposed in or a component of a cell cultivation system. The cells may be cultured (e.g., on the cell-culture substrates, growth substrates, and/or disk stacks) for various lengths of time as provided herein. In certain cases, the methods may include incubating the cells in a growth medium. The growth medium may be disposed in a bioreactor or a cell cultivation system.

Culture conditions as described herein (e.g., growth medium components, temperature, pH, etc.) may be varied. The cells may be cultured or incubated for an amount of time (e.g., a predetermined amount of time) as discussed herein. The methods may include rotating the cells through the growth medium. The methods may include incubating the cells. The cells may be muscle cells, connective tissue cells, fat cells, stem cells, mesenchymal cells, vascular cells, blood-associated cells, liver cells, nervous system cells, or cells from any edible animal organ, etc. The cells can be derived from any suitable animal (e.g., a mammal, bird, reptile, amphibian, fish, invertebrates (e.g., insects, mollusks, etc.), etc.). For example, the cells may be bovine, porcine, duck, chicken, alligator, frog, salmon, shark, grasshopper, clam, etc. The methods may include harvesting the cells. The methods may include using the cells (e.g., the harvested cells) for the formation of food products as provided herein. The food products may be for human and/or animal consumption (e.g., cell-based meat and cell-based fat). Cell-based meats and cell-based fats may include one type of cell or a combination of types of cells. To form or generate the cell-based meats and/or the cell-based fats, the harvested cells may be processed. For example, the cells may be centrifuged (e.g., to reduce water content) and/or the cells may be combined or compounded with other edible ingredients or materials (e.g., texturizers, stabilizers, etc.).

While preferred embodiments of the present disclosure have been shown and described herein, it will be obvious to those skilled in the art that such embodiments are provided by way of example only. Numerous variations, changes, and substitutions will now occur to those skilled in the art without departing from the systems and methods of the present disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. (canceled)

2. A cell cultivation system comprising:
at least one growth substrate for growing one or more cell types, wherein the at least one growth substrate has a mesh porosity and a surface area, and wherein cell growth density per unit area of the at least one growth substrate increases with: 1) increasing mesh porosity of the at least one growth substrate, or 2) decreasing surface area per unit area of the at least one growth substrate.

3. The system of claim 2, wherein the mesh porosity and the surface area per unit area are based on a number of openings in the at least one growth substrate, and wherein the openings are arranged in a repeating pattern across a surface of the at least one growth substrate.

4. The system of claim 3, wherein the cell growth density per unit area on the at least one growth substrate increases with the number of openings by at least 5% when the number of openings is increased by at least 10%.

5. The system of claim 2, wherein the cell growth density per unit area on the at least one growth substrate increases by at least 5% when the surface area per unit area is decreased by at least 10%.

6. The system of claim 2, further comprising at least one spacer substrate adjacent to the at least one growth substrate, wherein the at least one spacer substrate is used to facilitate transport and distribution of a growth medium over or across a surface of the at least one growth substrate.

7. A cell cultivation system comprising:
at least one growth substrate for growing one or more cell types; and
at least one spacer substrate adjacent to the at least one growth substrate, wherein the at least one spacer substrate is sized to influence a cell growth density per unit area on the at least one growth substrate such that a higher cell growth density per unit area is achieved on the at least one growth substrate when using a smaller spacer substrate as compared to a larger spacer substrate.

8. The system of claim 7, wherein a size of the at least one spacer substrate is based on a dimension as measured along a radial or longitudinal direction of the at least one spacer substrate, and wherein the cell growth density per unit area on the at least one growth substrate increases as the dimension of the at least one spacer substrate is reduced along the radial or longitudinal direction.

9. The system of claim 7, wherein the cell growth density per unit area on the at least one growth substrate increases by at least 5% when the size of the at least one spacer substrate is increased by at least 10%.

10. The system of claim 7, wherein the smaller spacer substrate has a smaller surface area than the larger spacer substrate, and wherein the smaller spacer substrate has a same thickness as the larger spacer substrate.

11. The system of claim 7, wherein a size of the at least one growth substrate is substantially equal to or greater than a size of the at least one spacer substrate.

12. The system of claim 7, wherein the at least one growth substrate and the at least one spacer substrate comprise a same number of openings per unit area.

13. The system of claim 7, wherein the at least one growth substrate and the at least one spacer substrate each comprise a different number of openings per unit area.

14. A bioreactor comprising:
a container configured to retain a growth medium;
a rotatable shaft disposed in the container;
a first growth substrate and a second growth substrate, wherein the first growth substrate and the second growth substrate are coupled to the rotatable shaft and configured to grow a biological material; and
a first spacer substrate disposed between the first growth substrate and the second growth substrate.

15. The bioreactor of claim 14, wherein a growth surface on the first growth substrate or second growth substrate is substantially planar.

16. The bioreactor of claim 14, wherein each of the first growth substrate and the second growth substrate comprise a mesh formed from a plurality of wires.

17. The bioreactor of claim 16, wherein adjacent wires of the plurality of wires are separated by a distance of 1 μm to 5,000 μm.

18. The bioreactor of claim 14, wherein the first growth substrate and the second growth substrate are porous and have a porosity of 0.001 to 0.999.

19. The bioreactor of claim 14, wherein the first growth substrate is disposed 1 μm to 5,000 μm from the first spacer substrate.

20. The bioreactor of claim 14, further comprising a plurality of growth substrates and a plurality of spacer substrates, wherein the plurality of growth substrates are arranged substantially parallel to the plurality of spacer substrates.

21. The bioreactor of claim 14, wherein the biological material comprises cells, tissues, organs, cell-based meat, cell-based fat, or a combination thereof.

22. The bioreactor of claim 14, wherein the rotatable shaft is coupled to a lateral center of the first growth substrate and to a lateral center of the second growth substrate.

23. The bioreactor of claim 20, wherein a ratio of an area of the plurality of growth substrates to an area of the plurality of spacer substrates is 1000:1 to 1:1.

24. The bioreactor of claim 20, wherein the plurality of growth substrates have an average pore size greater than an average pore size of the plurality of spacer substrates.

25. The bioreactor of claim 20, wherein the container comprises a hermetically sealable container.

26. The bioreactor of claim 20, wherein the plurality of growth substrates are formed from a first material, and wherein the plurality of spacer substrates are formed from a second material, the first material being different than the second material.

27. The bioreactor of claim 14, wherein the growth substrates comprise a coating that selectively adheres to the cells, wherein the coating comprises a cell-adhesive protein, a cell-adhesive peptide, a coating derived from an animal cell extracellular matrix, collagen, fibronectin, laminin, poly-L-lysine, poly-D-lysine, a coating derived from a plant, arginine-glycine-aspartic acid (RGD)-containing vitronectin-like protein, or a combination thereof.
